(19) United States
(12) Patent Application Publication
(10) Pub. No.: US 2009/0215025 A1
(43) Pub. Date: Aug. 27, 2009

(54) SMALL PEPTIDIC AND PEPTIDO-MIMETIC AFFINITY LIGANDS FOR FACTOR VIII AND FACTOR VIII-LIKE PROTEINS

(75) Inventors: Sebastian Knor, Munich (DE); Horst Kessler, Munich (DE); Charlotte Hauser, Munich (DE); Alexey Khrenov, North Potomac, MD (US); Evgueni Saenko, Moscow (RU)

Correspondence Address: LAHIVE & COCKFIELD, LLP;FLOOR 30, SUITE 3000, ONE POST OFFICE SQUARE, BOSTON, MA, 02109, US

(73) Assignee: TECHNISCHE UNIVERSITAT MUNCHEN, Munich (DE)

(21) Appl. No.: 12/096,503

(22) PCT Filed: Dec. 7, 2006
(86) PCT No.: PCT/EP2006/011786

§ 371 Date: Mar. 11, 2009

Related U.S. Application Data
(60) Provisional application No. 60/748,303, filed on Dec. 7, 2005.

(51) Int. Cl. C12Q 1/00; C07K 5/117; C07K 7/06; C07K 5/113; C07D 209/20; C12N 9/96

(52) U.S. Cl. 435/4; 530/330; 530/329; 530/331; 548/491; 525/54.1; 435/188

(57) ABSTRACT

The present invention pertains to compounds that show a high affinity to Factor VIII and Factor VIII-like proteins, and their uses. The compounds are characterized by the general Formula B-Q-X, wherein B represents a dipeptide, tripeptide, or a peptidomimetic; Q represents a spacer and X represents an anchoring molecule; Q and X are optional. These compounds can be used for coating a solid support material. The resulting coated solid support material can be used for purifying Factor VIII by means of affinity chromatography methods. In addition, the compounds of the present invention may be used for stabilizing Factor VIII and for enhancing its activity. The present invention thus pertains also to methods for manufacturing a stabilized Factor VIII containing medicament of increased activity. The inventive compounds can furthermore be used in diagnostic kits and as research tools.

### FIELD OF THE INVENTION

The present invention is related to the composition of small molecules and their use in the field of protein isolation and purification.

In particular, the present invention relates to the synthesis and optimization of compounds comprising small peptides and peptido-mimetics with affinity to coagulation Factor VIII and/or Factor VIII-like polypeptides. These compounds are useful for labeling, detecting, identifying, isolating and preferably for purifying of Factor VIII and Factor VIII-like polypeptides from physiological and non-physiological solutions comprising same. Further, these compounds may be used as ligands, which bind Factor VIII and Factor VIII-like polypeptides in methods of the present invention.

For the purpose of the present invention all references as cited herein are incorporated by reference in their entireties.

### BACKGROUND

Factor VIII (FVIII) is an essential component of the intrinsic pathway of blood coagulation (Bolton-Maggs, P. H. B.; K. J. Pasi Lancet 2003, 361, 1801). This plasma protein is circulating in blood in complex with von Willebrand factor (vWf), which protects and stabilizes it. Genetic deficiency of FVIII function results in a life-threatening bleeding disorder known as Hemophilia A, one of the most common bleeding disorders, which is treated by repeated infusions of FVIII. Hemophilia A is the result of an inherited deficiency of Factor VIII. For medical treatment, patients are given doses of Factor VIII derived from either blood plasma or recombinant cells.

Hemophilia A, the hereditary X chromosome-linked bleeding disorder caused by deficiency or structural defects in a coagulation Factor VIII (FVIII), affects approximately one in 5000 males. The clinical severity of Hemophilia A correlates with the degree of factor deficiency and is classified as severe disease with FVIII levels of less than 1%, moderate (1-5% FVIII levels) and mild disease (5-25% FVIII levels). The disease is characterized by spontaneous bleedings, as well as by uncontrollable bleedings in case of trauma or surgery. Other clinical hallmarks of Hemophilia A are acute recurrent painful hemarthrosis, which can progress to chronic arthropathy characterized by progressive destruction of the cartilage and the adjacent bone, muscle hematoma, intracerebral hemorrhages and hematuria (Klinge, J.; Ananyeva, N. M.; Hauser, C. A.; Saenko; E. L. Semin. Thromb. Hemost. 2002, 28, 309-322).

Hemophilia A is treated by repeated infusions of FVIII, derived from either human blood plasma or recombinant cells, expressing FVIII.

The FVIII molecule (˜300 kDa, 2332 amino acid residues) consists of three homologous A domains, two homologous C domains and the unique B domain, which are arranged in the order of A1-A2-B-A3-C1-C2. Prior to its secretion into plasma, FVIII is processed intracellularly to a Me²⁺-linked heterodimer produced by cleavage at the B-A3 junction. This cleavage generates the heavy chain (HCh) consisting of the A1 (1-372), A2 (373-740) and B domains (741-1648) and the light chain (LCh) composed of the A3 (1690-2019), C1 (2020-2172) and C2 (2173-2332) domains. The resulting protein is heterologous in size due to a number of additional cleavages within the B domain, giving the molecules with B-domains of different length. The C-terminal portions of the A1 (amino acids 337-372) and A2 (amino acids 711-740) domains and the N-terminal portion of LCh (amino acids 1649-1689) contain a high number of negatively charged residues and are called acidic regions (AR1, AR2 and AR3, respectively).

In order to cope with existing demands for better supply of FVIII as well as to reduce the risk of viral and prion contamination, the use of recombinant FVIII has been drastically increased in the past years (Ananyeva N., Khrenov A., Darr F., Summers R., Sarafanov A., Saenko E. Expert Opin. Pharmacother. 2004; 5:1061-1070). Since the isolation of the Factor VIII gene in 1984 (Vehar, G. A.; Keyt, B.; Eaton, D.; Rodriguez, H.; O'Brien, D. P.; Rotblat, F.; Oppermann, H.; Keck, R.; Wood, W. I.; Harkins, R. N. Nature 1984, 312, 337-342; Toole, J. J.; Knopf, J. L.; Wozney, J. M.; Sultzman, L. A.; Buecker, J. L.; Pittman, D. D.; Kaufman, R. J.; Brown, E.; Shoemaker, C.; Orr, E. C. Nature 1984, 312, 342-347), preparations of novel recombinant Factor VIII molecules have greatly improved. Moreover, deeper insights in structure-function relationship of Factor VIII as well as more sophisticated techniques in molecular biology have opened up new possibilities in the generation of recombinant Factor VIII.

Several therapeutic recombinant FVIII products are currently available as lyophilized concentrates.

(1) For the synthesis of RECOMBINATE (Baxter) and BIOCLATE (Centeon), the genes for FVIII and vWf have been inserted into Chinese hamster ovary cells (CHO). The vWf acts as stabilizer for FVIII in cell culture. The recombinant protein is purified by single immunoaffinity chromatography using a murine monoclonal antibody followed by two ion exchange chromatography steps to complete the purification process. The purified recombinant FVIII is finally stabilized by the addition of pasteurized human albumin. The purification process does not include a separate virus inactivation step (Kaufman, R. J.; Wasley, L. C.; Furie, B. C.; Furie, B.; Shoemaker, C. B. J. Biol. Chem. 1986, 261, 9622-9628).

(2) In the case of KOGENATE (Bayer) and HELIXATE (Centeon), the gene for Factor VIII has been inserted into an established cell line from baby hamster kidney (BHK). The secreted recombinant FVIII is processed by multiple purification steps, including two ion-exchange chromatography gel filtration and size exclusion chromatography, as well as double immunoaffinity chromatography using a murine monoclonal antibody. The purified protein is then stabilized by pasteurized human albumin. Virus inactivation is achieved by heat-treatment (Addiego, J. E. Jr.; Gomperts, E.; Liu, S. L.; Bailey, P.; Courter, S. G.; Lee, M. L.; Neslund, G. G.; Kingdon, H. S.; Griffith, M. J. Thrombosis and haemostasis 1992, 67, 19-27).

(3) KOGENATE FS (Bayer) has been developed as a second generation product. Different to KOGENATE, KOGENATE FS is cultured in cell culture medium containing recombinant insulin and Human Plasma Protein Solution (HPPS), but no proteins derived from animal sources.

(4) REFACTO (Wyeth-Ayerst Pharmacia and Upjohn) is the first licensed B domain deleted recombinant FVIII molecule (BDDrFVIII). The r-FVIII SQ gene which encodes a single chain 170 kDa polypeptide, was derived from full-length cDNA by removing the major part of the region encoding the B-domain. The r-FVIII SQ vector system was inserted into CHO cells and cultured in a serum-free medium supplemented with human albumin and recombinant insulin. The purification comprises five different chromatography steps including immunoaffinity with monoclonal antibodies directed to the heavy chain of FVIII, and a chemical solvent/detergent virus inactivation step (Eriksson, R. K.; Fenge, C.; Lindner-Olsson, E.; Ljungqvist, C.; Rosenquist, J.; Smeds, A. L.; Ostlin, A.; Charlebois, T.; Leonard, M.; Kelley, B. D.; Ljungqvist, A. Sem. Hematol. 2001, 38, 24-31).

It is important in the development of FVIII products to avoid any use of animal or human proteins in order to improve safety. In contrast to currently licensed recombinant FVIII preparations, next generation FVIII products will adapt production methods to culture media that do not contain any components of human or animal origin. Thus, the ultimate goal is an improvement in the production of FVIII to the point of completely avoiding any contact with components derived from animal or human raw materials. There is also a demand to improve purification methods for FVIII. Methods offering FVIII of high purity and activity obtained directly from various solutions such as blood or cell culture supernatants remain in demand, thereby, reducing the number of purification steps, and cost involved. New methods to gain FVIII in a faster, more efficient and cost-effective way remain unrealized by the current art.

Factor VIII is usually concentrated by affinity chromatography, employing monoclonal antibodies as ligands (Amatschek, K; Necina, R.; Hahn, R.; Schallaun, E.; Schwinn, H.; Josic, D.; Jungbauer, A. J. High Resol. Chromatogr. 2000, 23, 47-58). According to recent statements of the Medical and Advisory Council of the US National Hemophilia Foundation and of the World Federation of Hemophilia, all efforts should be made to eliminate human and bovine proteins from the manufacturing process of recombinant products (Medical and Scientific Advisory Council (MASAC) document #151 <<(MASAC RECOMMENDATIONS CONCERNING THE TREATMENT OF HEMOPHILIA AND OTHER BLEEDING DISORDERS>>, available at National Hemophilia Foundation website).

Use of oligo- and polypeptides as the partners of affinity ligands polypeptides has been suggested (see, WO 9914232; or US 2003165822, each incorporated herein by reference in their entirety). Nevertheless, this method still has disadvantages. First, the large scale synthesis and purification of oligopeptides is not trivial and quite cost-intensive. Furthermore, oligopeptides are sensitive towards proteolytic degradation and the presence of proteases cannot be completely avoided if raw materials derived from blood or cell cultures are applied to the affinity column. This may rapidly lead to inefficiency and reduced selectivity of the affinity purification step and, furthermore, to a reduced purity and half life of the eluted factor samples as well as half life of the expensive column material.

Thus, different to currently commercially available recombinant FVIII preparations the present invention is directed to compounds and methods for preparing a next generation product in culture media devoid of any components of human or animal origin. Furthermore, the present invention may also be directed to purification of plasma-derived FVIII preparations.

Our invention includes compounds comprising chemically synthesized unique high-affinity peptides and peptido-mimetics which can replace monoclonal antibodies and have improved proteolytic stability compared to the known oligopeptides mentioned above. Furthermore, our compounds are suitable for large scale solution synthesis and therefore will minimize the production costs.

### SUMMARY OF THE INVENTION

The present invention is directed to specific compounds comprising peptides and/or peptido-mimetics. These compounds exhibit particular properties of binding and/or releasing FVIII or FVIII-related polypeptides and may serve as ligands for affinity separation of FVIII or FVIII-related polypeptides.

In specific embodiments the compounds of the present invention comprising peptides and/or peptido-mimetics are dipeptides, tripeptides or peptido-mimetics that bind FVIII and/or FVIII-related proteins with affinity, sufficient for chromatographic purification of FVIII.

In certain embodiments, the compounds are binding molecules that exhibit distinct characteristics for binding of the target Factor VIII polypeptides as well as specific characteristics for release (elution) of the target polypeptides (i.e. specific composition and pH of application and elution buffers). To facilitate elution of the product under mild conditions, the compounds may easily be modified by existing chemical methods. Such modification is not technically feasible for the conventionally used antibodies.

A further embodiment relates to an inert matrix as support material comprising the immobilized compound, preferably a peptide and/or peptido-mimetic. In specific embodiments, the support material is a polymeric material. In further specific embodiments, the compound is chemically bound to the support matrix. In another specific embodiment, the compound is chemically bound to the support matrix via an anchoring molecule. In a further specific embodiment, the compound is chemically bound to the support matrix via a spacer molecule. It is also contemplated that the compound is chemically bound to the support matrix through an anchoring molecule and an additional spacer molecule.

The present invention relates to a diagnostic device or kit comprising a compound of the present invention immobilized on a matrix, wherein the compound binds specifically to a FVIII or FVIII-related protein. In certain embodiments, the compound is directly or via an anchoring compound and/or a spacer molecule immobilized on the matrix, which may be a polymeric material such as, for example, a resin.

In yet another embodiment, the compounds are used in methods as a label of a FVIII or FVIII-related protein.

In an embodiment of the present invention, the compound is used in methods of identification and/or purification of FVIII or FVIII-related proteins.

The present invention relates further to the medical use of the compound of the present invention in the treatment of diseases.

### DESCRIPTION OF THE FIGURES

FIG. 1 shows a purification profile for purification of previously purified FVIII, as described in Example 3.

FIG. 2 depicts a purification profile for FVIII from cell-conditioned, FBS-containing media spiked with FVIII, as described in Example 3.

FIG. 3 is a photograph of an SDS-PAGE of fractions form the adsorption and elution of pure FVIII (Lanes 2-6) and from the purification from cell-conditioned FBS-containing media spiked with FVIII (Lanes 7-12), as described in Example 3. Lane 1: molecular weight standards; Lane 2: pure FVIII; Lane 3: source solution with pure FVIII for the column; Lane 4: flow-through; Lane 5 and 6: elution fractions; Lane 7: media; Lane 8: media with FVIII for column; Lane 9: flow-through; Lane 10 and 11: elution fractions from pre-wash with 0.2 M NaCl; Lane 12: elution fraction with 1 M NaCl.

FIG. 4 is the photograph of a Western Blot of fractions form the adsorption and elution of pure FVIII (Lanes 2-6) and from the purification from cell-conditioned FBS-containing media spiked with FVIII (Lanes 8-12), as described in Example 3. Lane 1: pure FVIII; Lane 2: source solution with pure FVIII for the column; Lane 3: flow-through; Lane 4 and 5: elution fractions; Lane 6: media with FVIII for column; Lane 7: flow-through; Lane 8 and 9: elution fractions from pre-wash with 0.2 M NaCl; Lane 10: elution fraction with 1 M NaCl.

FIG. 5(a) shows the purification profile for purification of previously purified FVIII, as described in Example 4.

FIG. 5(b) is a photograph of an SDS-PAGE of fractions from the purifications from previously purified FVIII (Lanes 1-4), as described in Example 4. Lane 1: pure FVIII; Lane 2: flow-through; Lane 3: wash fraction; Lane 4: elution fraction with 1 M NaCl.

FIG. 5(c) shows a photograph of a Western Blot analysis of fractions from the purifications from previously purified FVIII, as described in Example 4. Lane 5: pure FVIII; Lane 6: flow-through; Lane 7: elution fraction with 1 M NaCl.

FIG. 6(a) shows the purification profile for purification from FBS-containing DMEM conditioned medium, spiked with FVIII, as described in Example 4.

FIG. 6(b) is a photograph of an SDS-PAGE of fractions from the purifications from FBS-containing DMEM conditioned medium, spiked with FVIII (Lanes 1-4), as described in Example 4. Lane 1: crude medium; Lane 2: source solution for the column; Lane 3: flow through; Lane 4: elution fraction with 1 M NaCl.

FIG. 6(c) is a photograph of a Western Blot analysis of fractions from the purifications from FBS-containing DMEM conditioned medium, spiked with FVIII, as described in Example 4. Lane 5: pure FVIII; Lane 6: flow-through; Lane 7: elution fraction with 1 M NaCl.

### DETAILED DESCRIPTION OF THE INVENTION

The affinity chromatography is a well established powerful technique which is a state-of-the-art procedure used for purification of complex molecules such as proteins (Jack, G. W.; Beer, D. J. Methods Mol. Biol. 1996, 59, 187-196). Affinity chromatography offers the unique possibility to isolate the target protein with excellent selectivity from contaminating proteins by its strong interaction between a target molecule and a ligand, which is immobilized on a resin. Usually, the ligands are either polyclonal or monoclonal antibodies. Monoclonal antibodies are preferred, because they are monospecific and can be produced with precision (Scopes, R. K. Protein purification: Principles and Practice. Springer, N.Y., 1994). Small chemical ligands had so far only limited application in affinity separation. However, the use of combinatorial libraries has expanded the repertoire of immunoaffinity chromatography techniques for peptide ligands (Lowe, C. R. Curr. Opin. Chem. Biol. 2001, 5, 248-256). Based on either biological or chemical systems the use of the combinatorial methods have generated unique peptides which provide moderate or even high binding affinity to capture the target protein and elute it under mild conditions.

Huang, Ping Y. et al., for example, describe in “Bioorganic & Medicinal Chemistry”, Vol. 4, No. 5, pp. 699-708, 1996 the use of immobilized peptides for the immunoaffinity chromatography purification of von Willebrand Factor (vWF). Purification of multimeric vWF is a major challenge because the molecular weight ranges between the enormous size of 0.5 to 10 million Daltons. The von Willebrand Factor is a multifunctional plasma protein directly involved in the blood coagulation cascade. It has a prominent role in the events that lead to normal arrest of bleeding. Interactions between vWF and FVIII result in stabilizing and transporting FVIII. Two high affinity binding sites ensure efficient capture (Sadler. J. E.; Mannucci. P. M.; Berntorp. E.; Bochkov. N.; Boulyjenkov. V.; Ginsburg. D.; Meyer. D.; Peake. I.; Rodeghiero. F.; Srivastava. A. Thromb. Haemost., 2000, 84, 160-174).

FVIII is a large and complex protein which plays an important function in the blood coagulation cascade and has great therapeutic significance. Deficiencies in FVIII production in vivo caused by genetic mutations can lead to hemophilia which is treated by infusion of purified preparations of human FVIII (Lee, C. Thromb. Haemost. 1999, 82, 516-524). The current sources of human FVIII for treatment of hemophilic patients are plasma-derived FVIII and recombinant FVIII, the latter synthesized in Chinese hamster ovary (CHO) cells (Kaufman, R. J.; Wasley, L. C.; Dorner, A. J. J. Biol. Chem. 1988, 263, 6352-6362) and baby hamster kidney (BHK) cells (Boedeker, B. D. G. Sem. Thromb. Hemost. 2001, 27, 385-394). In addition to high purity criteria, it is critical to ensure immunological and virus safety.

There are several methods known for purification of human FVIII using chromatographic techniques. The use of an immunoaffinity chromatography resin is a common manufacturing procedure for all recombinant FVIII preparations, and for many plasma-derived FVIII products. The current manufacturing process which includes affinity chromatography uses a monoclonal antibody (mAb) that is specific for FVIII (Lee, C., Recombinant clotting factors in the treatment of hemophilia. Thromb. Haemost. 1999, 82, 516-524). For manufacturing purposes the antibody against FVIII is produced by a murine hybridoma cell line and then immobilized to a chromatographic resin. The current industrial FVIII purification utilizes a mAb immunoaffinity step providing excellent removal of process-related impurities such as DNA and host cells.

However, there are a variety of concerns and limitations connected with the current process of immunoaffinity chromatography using immobilized monoclonal antibodies. One disadvantage is a limitation in the capacity of the resin because a few antibody molecules, huge in molecular size, will cover a considerable part of the resin surface. In addition, the antibody preparation is a lengthy and expensive method, and the purity and activity of the antibodies vary depending on each iterative preparation. The antibodies are produced by a hybridoma culture as the production host which makes the antibodies susceptible to a low, but non-zero risk that viruses, especially retroviruses, may be introduced into the manufacturing process of the target protein. Finally, the leakage of the antibodies from the support matrix, i.e. the resin, can lead to serious product contaminations and result in the loss of the product due to immunogenicity. Thus, there is sufficient motivation to replace the current process by a more precise, cost-effective process. The present invention fulfills this need by providing a compound which is a chemically synthesized small peptide or peptido-mimetic derivative in an immunoaffinity chromatography purification method that offers a reduction or elimination of several of the described pitfalls.

Pflegerl et al. (J. Peptide Res. 2002, 59, 174-182) reported the development of different octapeptides with high affinity towards FVIII. The essential amino acid sequence was found to be WEY, located in the C-terminal side of the peptides.

The compounds of this invention comprising small peptides or peptido-mimetic derivatives have advantages as ligands, because they are unlikely to provoke immune responses in case of leakage into the product. Small peptides or peptido-mimetic derivatives are also much more stable in comparison with antibodies. Another advantage is their significant lower production costs, since they can easily be manufactured aseptically in huge quantities under good manufacturing practices (GMP). The use of the small peptides or peptido-mimetic derivatives and methods of the present invention achieve a purified product using no animal-derived or human-derived raw materials. Last but not least, the sophisticated chemical synthesis described herein allows refined steps to improve the affinity of the small peptides or peptido-mimetic derivatives towards their target protein.

Therefore, the present invention provides the ordinary artisan working in the field with a compound and a process that improves the commonly used purification procedure of FVIII.

As described herein, the present invention provides a FVIII purification method that avoids the use of mouse monoclonal antibodies for immunoaffinity purification of FVIII. The invention includes chemically synthesized unique high-affinity peptides and peptido-mimetics which can replace monoclonal antibodies and have improved proteolytic stability compared to the known oligopeptides mentioned above. This would meet the up-to-date requirements for biological safety. Furthermore, our compounds are suitable for large scale solution synthesis and therefore minimize the production costs of the affinity ligands.

The present invention comprises novel compounds, preferably dipeptides, tripeptides and peptido-mimetics as ligands for detecting, identifying, isolating and purifying as well as labeling active Factor VIII and Factor VIII-like proteins from solutions that contain such proteins. The Factor VIII binding molecules of the present invention exhibit remarkable stability as well as high affinity for Factor VIII and Factor VIII-like proteins.

Unless otherwise specified or indicated, as used herein, the terms “Factor VIII and Factor VIII-like proteins” encompass any Factor VIII protein molecule from any animal, any recombinant or hybrid Factor VIII or any modified Factor VIII. In a preferred embodiment, such “Factor VIII and Factor VIII-like proteins” are characterized by an activity (as determined by the standard one stage clotting assay, as described e.g., in Bowie, E. J. W., and C. A. Owen, in Disorders of Hemostasis (Ratnoff and Forbes, eds.) pp. 43-72, Grunn & Stratton, Inc., Orlando, Fla. (1984)), of at least 10%, more preferably at least 50%, most preferably at least 80%, of the activity of native human form of Factor VIII.

Factor VIII-like proteins also encompass domains, fragments and epitopes of factor VIII proteins of any source, as well as hybrid combinations thereof. The term “Factor VIII-like proteins” furthermore includes fragments of Factor VIII, which can be used as probes for research purposes or as diagnostic reagents even though such fragments may show little or no blood clotting activity. Such proteins or polypeptides preferably comprise at least 50 amino acids, more preferably at least 100 amino acids. Preferred domains, epitopes and fragments of Factor VIII and Factor VIII-like proteins include the light chain thereof, parts of the light chain containing the domains A3-C1, C₁-C₂, A3, C1, or C2 and the individual domains A3, C1 and C2. The Factor VIII and Factor VIII-like proteins that can be purified according to the present invention also include all recombinant proteins, hybrids, derivatives, mutants, domains, fragments, and epitopes described in U.S. Pat. No. 7,122,634, U.S. Pat. No. 7,041,635, U.S. Pat. No. 7,012,132, and U.S. Pat. No. 6,866,848, all of which are incorporated herein by reference in their entirety. Unless specified otherwise herein, the term “amino acid” encompasses any organic compound comprising at least one amino group and at least one acidic group. The amino acid can be a naturally occurring compound or be of synthetic origin. Preferably, the amino acid contains at least one primary amino group and/or at least one carbocylic acid group. The term “amino acid” also refers to residues contained in larger molecules such as peptides and proteins, which are derived from such amino acid compounds and which are bonded to the adjacent residues by means of peptide bonds or peptido-mimetic bonds.

The invention provides a cost-effective means to ensure fast separation and purification of commercial quantities of proteins and related substances useful in the treatment and research of hemophilia A.

The invention relates to compounds comprising peptides and/or peptido-mimetic of formula I

B-Q-X (I)
where
B is a dipeptide, tripeptide or peptido-mimetic providing affinity to FVIII and/or FVIII-like proteins,
Q is missing or is an organic spacer molecule and
X is missing or is an anchorage molecule,
   as well as their salts. Further compounds in accordance with the present invention comprise two or more groups B, which may be the same or different from each other. These groups B can be attached to the same spacer Q, to thereby form a compound represented by the general formula (B)r-Q-X with r ranging from 2 to 4. Alternatively, they can be connected to each other by means of further spacers Q (the individual spacers Q being the same or different from each other) to thereby form an oligomeric compound of the type (B-Q)s-X, with s ranging from 2 to 4.

According to another embodiment of the present invention, the group B as such or groups B and Q together or B and X together or B and Q and X together may form a cycle. Optionally, this cycle may include a further ring-forming moiety that is selected from organic bivalent groups such as optionally substituted alkylene groups, amino acids, di- or tripeptides, and combinations thereof.

The term “peptido-mimetic” comprises compounds containing non-peptidic structural elements which are capable of mimicking or antagonizing the biological action(s) of a parent peptide. Such compounds preferentially comprise few (or no) peptide bonds. A preferred embodiment of the present invention relates to peptido-mimetics that are derived from the dipeptides and tripeptides of the present invention by replacing one or more peptide bonds by one or more functional groups selected from the group consisting of —CO—NR²—, —NR²—CO—, —CH₂—NR²— or —NR²—H₂—, —CO—CHR²—, —CHR²—CO—, —CR²═CR²— and —CR²═CR²—, wherein R² is as defined below with respect to general formula (II). It should be understood that in the options containing a group —NR²—, substituent R² may be the side chain of the respective amino acid (peptoid amino acid). In this case, the adjacent Cα does not carry the side chain. Other substituents R², on the other hand, are present in addition to the side chain attached to Cα. Moreover, if more than one R² is present, it should be understood that the individual R²'s can be the same or different from each other.

A particularly preferred group of peptido-mimetics comprises those compounds, which contain residues Z1-Z2-Z3 (as defined below), and wherein (at least) the peptide bond between Z2 and Z3 is selected from —CH₂—NR²— or —NR²—CH₂—, —CR²═CR²— and —CR²═CR²—,

“Affinity” is the force of attraction between atoms or molecules that helps to keep them in combination. This is the basis for affinity chromatography. In the context of the present invention, a peptide or peptidomimetic is considered to show affinity to FVIII or FVIII-like proteins if a binding to FVIII is measured according to the test protocol below, which is at least 10%, preferably at least 25% and most preferably at least 40%. The degree of binding is measured by reproducing the experiment described in Example 1 below using ¹²⁵I-labeled FVIII.

The peptide or peptido-mimetic derivative B of the present invention (as used herein, the terms “peptido-mimetic” and “peptido-mimetic derivative” are used interchangeably) can preferably be chemically bound to the surface of a support matrix, to thereby form a peptide-coated support matrix. This is preferably done with the help of an anchorage molecule X and/or a spacer molecule Q or, if Q and X are missing, preferably by a SH, N₃, NH—NH₂, O—NH₂, NH₂, —CH₂-L, C≡CH, carbonyl or carboxyl group of the compound B. Herein L comprises a leaving group, like Cl, Br or I.

The present invention also pertains to such peptide-coated support matrices.

The term “chemical binding” includes covalent, ionic, hydrophobic and/or other complex interactions, as well as mixtures and combinations thereof, between two (or more) atoms, or one (or more) atom(s) and one (or more) compound(s), or, two (or more) compounds.

The support material comprises inorganic or organic, especially polymeric, material. Therefore, the same polymeric material (i.e. linear polysaccharide) can be utilized which is usually employed for the chromatography of biopolymers. In particular, polymers exerting a hydrophilic surface are suitable as a chromatography support material, i.e. a resin. For example, the Toyoperal AF-Epoxy-650M resin is employed. Such support material can also be provided with an additional anchoring molecule offering, for example, a SH, N₃, NH—NH₂, O—NH₂, NH₂, —CH₂-L, C≡CH, epoxy, carbonyl or carboxyl group for immobilization of compounds like the peptides or peptido-mimetics according to formula I.

Due to the fact that the preferred compounds of the current invention interact with FVIII and/or FVIII-like proteins, the preferred compounds comprising peptides or peptido-mimetic derivatives are suitable for diagnostic devices and kits. The preferred diagnostic device or kit comprises at least one compound, having a high affinity for FVIII or FVIII-related proteins, a support matrix to which at least one compound may optionally be bound chemically, and other reagents, if needed.

In order to follow the results of a reaction of the compound, preferably a peptide or peptido-mimetic derivative, of the present invention with FVIII and/or FVIII-like proteins, the compound is labeled. There are several possibilities to label the compound, i.e. by using radioactive markers, by using fluorescent ligands, by using the avidine/steptavidine system, or, as is common in the ELISA technique, by using enzymes which provoke color reactions.

The present invention relates to compounds comprising peptides and peptido-mimetic derivatives which are suitable for labeling, detecting, identifying, isolating and/or purifying FVIII and/or FVIII-like proteins.

The abbreviations of amino acids given above and below stand for the residues of the following amino acids:
Abu 4-Aminobutyric acid
Aha 6-Aminohexanoic acid
Ala Alanine
Asn Asparagine
Asp Aspartic acid
Arg Arginine
Bpa p-Benzoylphenylalanine
Cys Cysteine
Dab 2,4-Diaminobutyric acid
Dap 2,3-Diaminopropionic acid
Gln Glutamine
Glp Pyroglutamic acid
Glu Glutamic acid
Gly Glycine
His Histidine
homo-Cys homo-Cysteine
homo-Phe homo-Phenylalanine
IAA 2-(Indol-3-yl)acetic acid
IBA 4-(Indol-3-yl)butyric acid
IPA 3-(Indol-3-yl)propionic acid
Ile Isoleucine
Leu Leucine
Lys Lysine
Met Methionine
1-Nal 1-Naphthylalanine
2-Nal 2-Naphthylalanine
Nle Norleucine
Orn Ornithine
Phe Phenylalanine
Phg Phenylglycine
4-Hal-Phe 4-Halogen-phenylalanine
Pro Proline
Ser Serine
Thr Threonine
Trp Tryptophan
Tyr Tyrosine
Val Valine

Furthermore, the following abbreviations are used below:
Ac Acetyl
BOC tert-Butoxycarbonyl
tBu tert-Butyl
CBZ oder Z Benzyloxycarbonyl
DCCl Dicyclohexylcarbodiimide
DIPEA N-Ethyldiisopropylethylamine
DMF Dimethylformamide
EDCl N-Ethyl-N,N-(dimethylaminopropyl)-carbodiimide
Et Ethyl
Fmoc 9-Fluorenylmethoxycarbonyl
HOBt 1-Hydroxybenzotriazole
Me Methyl
MBHA 4-Methyl-benzhydrylamine
Mtr 4-Methoxy-2,3,6-trimethylphenyl-sulfonyl
HATU O-(7-Azabenzotriazol-1-yl)-N,N,N′,N′-Tetramethyluronium-hexafluorophosphate
HONSu N-Hydroxysuccinimide
OtBu tert-Butylester
Oct Octanoyl
OMe Methylester
OEt Ethylester
POA Phenoxyacetyl
Pbf Pentamethylbenzofuranyl
Pmc 2,2,5,7,8-Pentamethylchroman-6-sulfonyl
Sal Salicyloyl
Su Succinyl
TIPS Triisopropylsilane
TFA Trifluoracetic acid
TFE Trimethylsilylbromide
Trt Trityl (Triphenylmethyl).

If the building blocks of the compounds of the formula I (i.e. the amino acids mentioned above) can occur in a plurality of enantiomeric forms, all these forms and also mixtures thereof (for example the DL forms) are included.

Furthermore, the amino acids may, for example, as a constituent of compounds of the formula I, be provided with corresponding protecting groups known per se. Favored groups are derivatives of Asp and Glu, particularly methyl-, ethyl-, propyl-, butyl-, tert-butyl-, neopentyl- or benzylester of the side chain or derivatives of Tyr, particularly methyl-, ethyl-, propyl-, butyl-, tert-butyl-, neopentyl- or benzylethers of the side chain. The compounds may furthermore carry one or more of the additional protecting groups that are described below in connection with the preparation of the compounds of the present invention.

In addition, also structural elements like N-terminal modified or carboxy-terminal modified derivatives are part of this invention. Favored groups are amino-terminal methyl-, ethyl-, propyl-, butyl-, tert-butyl-, neopentyl-, phenyl- or benzyl-groups, amino-terminal groups like BOC, Mtr, CBZ, Fmoc, and, particularly, acetyl, benzoyl or (indol-3-yl)carbonic acid groups, furthermore, carboxy-terminal methyl-, ethyl-, propyl-, butyl-, tert-butyl-, neopentyl- or benzylester, methyl-, ethyl-, propyl-, butyl-, tert-butyl-, neopentyl- or benzylamides and, particularly, carboxamides.

Alpha amino groups may be protected by a suitable protecting group selected from aromatic urethane-type protecting groups, such as allyloxycarbonyl, benzyloxycarbonyl (Z) and substituted benzyloxycarbonyl, such as p-chlorobenzyloxycarbonyl, p-nitrobenzyloxycarbonyl, p-bromobenzyloxycarbonyl, p-biphenyl-isopropyloxycarbonyl, 9-fluorenylmethyloxycarbonyl (Fmoc) and p-methoxybenzyloxycarbonyl (Moz); aliphatic urethane-type protecting groups, such as t-butyloxycarbonyl (Boc), diisopropylmethyloxycarbonyl, and isopropyloxycarbonyl. Herein, Fmoc is most preferred for alpha amino protection.

Amino acids, which can be used for the formation of the peptides and peptido-mimetics according to the present invention, can belong to both naturally occurring and non-proteinogenic amino acids. Amino acids and amino acid residues can be derivated, whereas N-methyl-, N-ethyl-, N-propyl- or N-benzyl-derivatives are favored. For example, if a methyl is employed, the N-alkylation of the amide binding can have a strong influence on the activity of the corresponding compound (Levian-Teitelbaum, D.; Kolodny, N.; Chorev, M.; Selinger, Z.; Gilon, C. Biopolymers 1989, 28, 51-64 which is herein incorporated by reference in its entirety). Further structural alternatives of amino acids that can be used include amino acids with modifications in the side chain, β-amino acids, aza-amino acids (derivatives of α-amino acids, where the αCH-group is substituted by a N-atom) and/or peptoid-amino acids (derivatives of α-amino acids, where the amino acid side chain is bound to the amino group instead to the α-C-atom) or cyclised derivatives from the above mentioned modifications.

According to one embodiment of the present invention, it is also possible to employ homo-derivatives of naturally occurring amino acids as building blocks. These are derivatives of the naturally occurring amino acids, wherein a methylene group is inserted into the side chain, immediately adjacent to Cα. Similarly, it is possible to use α-methylated derivatives of naturally occurring amino acids in accordance with the present invention. Above and below, the residues B, Q and X are as defined for the formula I, unless expressly stated otherwise.

In one embodiment of the present invention, the compound of Formula I is as defined in any one of the appended claims 2 to 17.

In another embodiment, B is preferably represented by the general formula

Z1-Z2-Z3,

wherein Q, X or the support may be bonded to any one of the residues Z1, Z2 and Z3. It is preferred that the binding is via residue Z1 or Z3, more preferably via residue Z3.

In this embodiment, Z1 is a natural occurring or non-proteinogenic amino acid residue or a derivative thereof with a large side chain. This means that the side chain comprises at least 3 carbon atoms, preferably at least 5 carbon atoms and more preferably from 6 to 25 carbon atoms. One or more of these carbon atoms may be replaced by a heteroatom selected from N, O and S. The side chain of Z1 contains preferably a cyclic group, which may be monocyclic, bicyclic or tricyclic. Moreover, this cyclic group may be saturated, unsaturated or aromatic. Aromatic groups are more preferred, as well as bicyclic groups. Aromatic bicyclic groups are particularly preferred. The features specified in appended Claims 4 to 17 for the other embodiment also characterize further preferred compounds of this embodiment.

In this embodiment Z1 may also preferably be a residue of the formula

Ar—(CH₂)ₘ—(CHR¹)ₙ—(CH₂)ₒ-A¹ (II)

wherein
A¹ represents a group selected from NR², CO, OCO, CHR², O or S,
R¹ represents a group selected from C₁₋₄ alkyl, phenyl, benzyl, and N(R²)₂, wherein the alkyl, phenyl or benzyl group may carry one or more substituents independently selected from A and N(R²)₂, wherein two or more A's and/or two or more R²'s may be the same or different from each other,
Ar is an aromatic group having a mono-, bi- or tricyclic aromatic ring system with 6 to 14 carbon atoms, a saturated or partially unsaturated C5-14 mono- or bicyclic alkyl group, each of which may be unsubstituted or carry one to three substituents independently selected from A, Ar¹, O—Ar¹, C(O)—Ar¹, CH₂—Ar¹, OH, OA, CF₃, OCF₃, CN, NO₂, Hal; or Ar may be Het,
Hal is selected from F, Cl, Br or I,
Ar¹ is an aromatic group having a mono-, bi- or tricyclic aromatic ring system with 6 to 14 carbon atoms, preferably a phenyl group or a naphthyl group, more preferably a phenyl group. Ar¹ may itself be unsubstituted or carry one to three substituents independently selected from A, OH, OA, CF₃, OCF₃, CN, NO₂, and Hal;
Het represents a saturated, partially or completely unsaturated mono- or bicyclic heterocyclic residue with 5 to 12 ring members, comprising 1 to 3 N- and/or 1 S- or O-atoms.
   Examples of heterocycles on which the heteroaryl radical or the radical of the monocyclic or bicyclic 5-membered to 12-membered heterocyclic ring can be based are pyrrole, furan, thiophene, imidazole, pyrazole, oxazole, isoxazole, thiazole, isothiazole, tetrazole, pyridine, pyrazine, pyrimidine, indole, isoindole, indazole, phthalazine, quinoline, isoquinoline, quinoxaline, quinazoline, cinnoline, β-carboline or benzo-fused, cyclopenta-fused, cyclohexa-fused or cyclohepta-fused derivatives of these heterocycles.
   Nitrogen heterocycles can also be present as N-oxides.
   Radicals which can be heteroaryl or the radical of a monocyclic or bicyclic 5-membered to 12-membered heterocyclic ring are, for example, 2- or 3-pyrrolyl, phenylpyrrolyl, for example 4- or 5-phenyl-2-pyrrolyl, 2-furyl, 3-furyl, 2-thienyl, 3-thienyl, 4-imidazolyl, methylimidazolyl, for example 1-methyl-2-, -4- or 5-imidazolyl, 1,3-thiazol-2-yl, 2-pyridyl, 3-pyridyl, 4-pyridyl, N-oxido-2-, -3- or -4-pyridyl, 2-pyrazinyl, 2-, 4- or 5-pyrimidinyl, 2-, 3- or 5-indolyl, substituted 2-indolyl, for example 1-methyl-, 5-methyl-, 5-methoxy-, 5-benzyloxy-, 5-chloro- or 4,5-dimethyl-2-indolyl, 1-benzyl-2- or -3-indolyl, 4,5,6,7-tetrahydro-2-indolyl, cyclohepta[b]-5-pyrrolyl, 2-, 3- or 4-quinolyl, 1-, 3- or 4-isoquinolyl, 1-oxo-1,2-dihydro-3-isoquinolyl, 2-quinoxalinyl, 2-benzofuranyl, 2-benzothienyl, 2-benzoxazolyl or 2-benzothiazolyl or, as radicals of partially hydrogenated or completely hydrogenated heterocyclic rings, for example also dihydropyridinyl, pyrrolidinyl, for example 2- or 3-(N-methylpyrrolidinyl), piperazinyl, morpholinyl, thiomorpholinyl, tetrahydrothienyl, benzodioxolanyl.
   Heterocyclic radicals representing the radical Het can be unsubstituted on carbon atoms and/or ring nitrogen atoms or monosubstituted or polysubstituted, for example disubstituted, trisubstituted, tetrasubstituted or pentasubstituted, by identical or different substituents. Carbon atoms can be substituted, for example, by (C₁-C₈)-alkyl, in particular (C₁-C₄)-alkyl, (C₁-C₈)-alkoxy, in particular (C₁-C₄)-alkoxy (the alkyl moiety of the aforementioned substituents may itself be unsubstituted or substituted with COOR² or N(R²)₂, halogen, nitro, N(R²)₂, trifluoromethyl, OCF₃, hydroxyl, oxo, cyano, COOR², aminocarbonyl, (C₁-C₄)-alkoxycarbonyl, phenyl, phenoxy, benzyl, benzyloxy, tetrazolyl, in particular by (C₁-C₄)-alkyl, for example methyl, ethyl or tert-butyl, (C₁-C₄)-alkoxy, for example methoxy, hydroxyl, oxo, phenyl, phenoxy, benzyl, benzyloxy. Sulfur atoms can be oxidized to the sulfoxide or to the sulfone. Examples of the radical Het are 1-pyrrolidinyl, 1-piperidinyl, 1-piperazinyl, 4-substituted 1-piperazinyl, 4-morpholinyl, 4-thiomorpholinyl, 1-oxo-4-thiomorpholinyl, 1,1-dioxo-4-thiomorpholinyl, perhydroazepin-1-yl, 2,6-dimethyl-1-piperidinyl, 3,3-dimethyl-4-morpholinyl, 4-isopropyl-2,2,6,6-tetramethyl-1-piperazinyl, 4-acetyl-1-piperazinyl, and 4-ethoxycarbonyl-1-piperazinyl.
A represents COOR², N(R²)₂ or a linear, branched or cyclic alkyl group with 1-6 C-atoms, which may be unsubstituted or be substituted with COOR² or N(R²)₂,
m and o are independently selected from 0, 1, 2, 3 and 4,
n is 0 or 1, and
R² is H, C₁₋₄ alkyl, phenyl or benzyl or, in the case of peptoid-amino acids, the amino acid side chain.

More preferred groups Z1 include proteinogenic aromatic amino acids (Phe, Tyr, Trp, His) and derivatives thereof, in particular those derivatives carrying one to three substituents selected independently from R⁶ (as defined below) at the side chain thereof. Typical examples of such derivatives are Tyr(OMe), Tyr(OBn), Trp(Me). More preferred groups Z1 include derivatives of Tyr, Tyr(OMe) and Tyr(OBn), wherein the substituent is attached to the meta- or otho-position of the phenyl group. Further groups Z1 that are more preferred include cyclohexylalanine, 1-naphthylalanine, 2-naphthylalanine, 2-thienylalanine, 3-thienylalanine, benzothienylalanine (wherein the bicyclic ring system can be attached to the remainder of the molecule at any position of the ring system, preferably at the 2- or 3-position of the thienyl ring), phenylglycine, p-benzoylphenylalanine, homophenylalanine, homotyrosine, homotryptophane, homohistidine and their derivatives as described above with respect to the natural amino acids.

Other more preferred groups Z1 include groups represented by the above general formula (II), which are represented by the following general formula (III):

Ar²—(CH₂)ₘ—(CHR³)ₙ—(CH₂)ₒ—CO— (III)

wherein
Ar² represents a preferred subgroup of the aromatic groups defined by Ar, including phenyl, 2-hydroxyphenyl, 3-hydroxyphenyl, 4-hydroxyphenyl, 1-naphthyl, 2-naphthyl, p-benzoylphenyl, (ortho-, meta-, or para-)biphenyl, 2-indolyl, 3-indolyl, 2-thiophenyl, 3-thiophenyl, 2-benzothiphenyl, 3-benzothiophenyl, each of which may carry one to three substituents independently selected from A and Hal, and
   wherein the remaining substituents of formula (III) are as defined with respect to formula (II) and
R³ is H, R⁶, —COR⁶, —COOR⁶ and
R⁶ represents H, C₁₋₄ alkyl, phenyl or benzyl, each of which may be unsubstituted or one-, two-, or threefold independently substituted with A, OH, OA, CF₃, OCF₃, CN, NO₂ or Hal.

Particularly preferred groups Z1 are selected from Ac-Trp, Trp, 1-naphthylalanine, 2-naphthylalanine, p-benzoylphenylalanine, and groups of the above general formula (III), wherein Ar represents 3-indolyl, 1-naphthyl, 2-naphthyl oder p-benzoylphenyl and m=0-3, n=0, o=0.

Most preferred are groups Z1 that are represented by general formula (III), wherein Ar means 3-Indolyl, m=1, n=0, o=0.

Z2 is missing or is a naturally occurring or non-proteinogenic amino acid residue or a derivative thereof. Preferably Z2 is not aromatic.

More preferably, Z2 is a polar amino acid including Ser, Thr, Glu, Asp, Asn, Gln, Arg, Lys, and derivatives thereof (including, for instance N-alkylated and Cα-methylated polar amino acids and polar amino acid derivatives with a modified side chain length such as homo-derivatives and Orn).

Particularly preferred groups Z2 are selected from polar amino acids as defined above, which carry a negative charge under physiological conditions, such as Glu, Asp, homo-Glu and homo-Asp. Most preferred groups Z2 are Glu or Asp.

Z3 is a residue as defined above for Z1. That is, Z3 is a naturally occurring or non-proteinogenic amino acid residue or a derivative thereof or a group represented by general formula (II), wherein
A¹ represents NR², CO, CHR², O, or S,
R¹ represents C₁₋₄ alkyl, phenyl or benzyl, and N(R²), wherein the alkyl, phenyl or benzyl group carries at least one group N(R²) and optionally one or more substituents independently selected from A and N(R²)₂, wherein two or more A's and/or two or more R²'s may be the same or different from each other, and
Ar is an aromatic group having a mono-, bi- or tricyclic aromatic ring system with 6 to 14 carbon atoms, a saturated or partially unsaturated C₅₋₁₄ mono- or bicyclic alkyl group, each of which may be unsubstituted or one-, two-, or threefold substituted with group independently selected from A, O—Ar1, C(O)—Ar¹, CH₂—Ar¹, OH, OA, CF₃, OCF₃, CN, NO₂ or Hal, or Het,
Hal is selected from F, Cl, Br or I,
Het is a heterocyclic residue as defined above with respect to Z1,
A represents COOR², N(R²)₂ or a linear, branched or cyclic alkyl group with 1-6 C-atoms, which may be unsubstituted or be substituted with COOR² or N(R²)₂
m and o are independently selected from 0, 1, 2, 3 and 4,
n is 0 or 1, and
R² is H, C₁₋₄ alkyl, phenyl or benzyl or, in the case of peptoid-amino acids, the amino acid side chain.

More preferred groups Z3 include proteinogenic aromatic amino acids (Phe, Tyr, Trp, His) and derivatives thereof, in particular those derivatives carrying one to three substituents selected independently from C₁₋₄ alkyl groups, halogen atoms or benzyl groups at the side chain thereof. Typical examples of such derivatives are Tyr(OMe), Tyr(OBn), Trp(Me). Further groups Z3 that are more preferred include cyclohexylalanine, 1-naphthylalanine, 2-naphthylalanine, thienylalanine, benzothienylalanine, phenylglycine, p-benzoylphenylalanine, homophenylalanine, homotyrosine, homotryptophane, homohistidine and their derivatives as described above with respect to the natural amino acids.

Other more preferred groups Z3 include groups represented by the above general formula (III), wherein Ar represents phenyl, 2-hydroxyphenyl, 3-hydroxyphenyl, 4-hydroxyphenyl, 1-naphthyl, 2-naphthyl, p-benzoylphenyl, biphenyl, 2-indolyl, 3-indolyl, thiophene, benzothiphene, each of which may carry one to three substituents independently selected from A and Hal, and wherein the remaining substituents of formula (III) are as defined above with respect to Z1.

Most preferred groups Z3 are selected from 1-Nal, Phe, Tyr and Tyr(OMe). The residues Z1 and Z3 or Z1 and Z2 as well as Z2 and Z3 in the dipeptide and tripeptide groups B of the present invention are each linked via a peptide bond.

Preferred compounds of the present invention include those di- and tripeptide groups B that are selected from the following combinations of residues:
(i) The combination of more preferred embodiments of Z1 with preferred embodiments of Z2 and Z3;
(ii) The combination of particularly preferred embodiments of Z1 with preferred embodiments of Z2 and Z3;
(iii) The combination of the most preferred embodiments of Z1 with preferred embodiments of Z2 and Z3;
(iv) The combination of more preferred embodiments of Z2 with preferred embodiments of Z1 and Z3;
(v) The combination of particularly preferred embodiments of Z2 with preferred embodiments of Z1 and Z3;
(vi) The combination of the most preferred embodiments of Z2 with preferred embodiments of Z1 and Z3;
(vii) The combination of more preferred embodiments of Z3 with preferred embodiments of Z1 and Z2;
(viii) The combination of the most preferred embodiments of Z3 with preferred embodiments of Z1 and Z2;
(ix) The combination of more preferred embodiments of Z1 with more preferred embodiments of Z2 and Z3;
(x) The combination of particularly preferred embodiments of Z1 with more preferred embodiments of Z2 and Z3;
(xi) The combination of the most preferred embodiments of Z1 with more preferred embodiments of Z2 and Z3;
(xii) The combination of more preferred embodiments of Z2 with more preferred embodiments of Z1 and Z3;
(xiii) The combination of particularly preferred embodiments of Z2 with more preferred embodiments of Z1 and Z3;
(xiv) The combination of the most preferred embodiments of Z2 with more preferred embodiments of Z1 and Z3;
(xv) The combination of more preferred embodiments of Z3 with more preferred embodiments of Z1 and Z2;
(xvi) The combination of the most preferred embodiments of Z3 with more preferred embodiments of Z1 and Z2;
(xvii) The combination of more preferred embodiments of Z1 with the most preferred embodiments of Z2 and Z3;
(xviii) The combination of particularly preferred embodiments of Z1 with the most preferred embodiments of Z2 and Z3;
(xix) The combination of the most preferred embodiments of Z1 with the most preferred embodiments of Z2 and Z3;
(xx) The combination of more preferred embodiments of Z2 with the most preferred embodiments of Z1 and Z3;
(xxi) The combination of particularly preferred embodiments of Z2 with the most preferred embodiments of Z1 and Z3;
(xxii) The combination of more preferred embodiments of Z3 with the most preferred embodiments of Z1 and Z2;

The direction of the peptide and/or peptido-mimetic sequence can be inverted (called a “retropeptide”).

Q refers to an optional organic spacer molecule.

Organic spacer molecules are known per se. “Organic” refers to all carbon compounds except carbide and carbonate compounds, see also Beilstein's Handbook of Organic Chemistry. Usually, the organic spacer molecule is a linear hydrocarbon having a functional groups at one or both terminal ends. The hydrocarbon chain can be modified. Preferred organic spacer molecules include amino acids or a [—NH—(CH₂)ₓ—CO]_{w}, [—NH—(CH₂CH₂—O—)_{y}CH₂—CO]_{w}, [CO—(CH₂)_{z}—CO—], [NH—(CH₂)_{z}—NH—], [CO—CH₂—(OCH₂CH₂)_{y}—O—CH₂—CO—] or [NH—CH₂CH₂—(OCH₂CH₂)_{y}—NH—] residue as well as combinations thereof. Indices w, x, y and z are respectfully 1-8; 1-5; 1-6; and 1-6. Furthermore, peptides, saccharides and other polyethers can act as organic spacer molecules.

X refers to an optional organic anchoring molecule.

Organic anchoring molecules are molecules or molecule-groups which can be applied for linking fragments (i.e. a compound and a resin). Such organic anchoring molecules are known per se. Usually organic anchoring molecules comprise two or more functional groups which can form a chemical binding.

Preferred organic anchoring molecules include a naturally occurring or non-proteinogenic amino acid or a -A¹-(CH₂)ₚ-A², -A¹-CH₂—(OCH₂CH₂)_{y}—O—CH₂-A², A1-CH₂CH₂—(OCH₂CH₂)_{y}-A², ═CR²—(CH₂)ₚ-A², -A¹-CH(NHR³)—(CH₂)_{q}-A², ═C R²—CH(NHR³)—(CH₂)_{q}-A², -A¹-CH(COR⁴) (CH₂)_{q}-A² or ═CR²—CH(COR⁴)—(CH₂)_{q}-A² residue.

A¹ is preferably NH but also CO, CHR², O or S and A² is preferably SH but also N₃, C≡CH, NH—NH₂, O—NH₂, NH₂, Hal¹, CR⁵O, or Carboxyl.

Furthermore
R² is as defined above,
R³ is as defined above with respect to Z1,
R⁴ is —OR⁶ or —NHR³,
R⁵ is H, C1-4 alkyl or unsubstituted or with A, OH, OA, CF₃, OCF₃, CN, NO₂ or Hal one-, two-, or threefold substituted phenyl or benzyl,
R⁶ is as defined above with respect to Z1,
p is 1-20,
q is 1-20,
y is 1-6,
z is 1-6,
Hal is as defined above,
Hal¹ is Cl, Br or I.

In preferred embodiments of the present invention, the groups -Q-X are characterized by one of the residues selected from -homo-Cys-OH, -Gly-Cys-OH, -Aha-Cys-OH, -Gly-Aha-Cys-OH and derivatives thereof. Preferred derivatives are moieties that contain a thiol group as well as a nitrogen atom that participates in the formation of a peptide bond with the adjacent residue (preferably Z3), and wherein said nitrogen atom and the sulfur atom of the thiol group are linked by a linear chain of from 2 to 14 atoms selected independently from C, N and O, Such derivatives are preferably unsubstituted or carry one to three substituents selected from R³ as defined above with respect to Z1.

The invention furthermore relates to the process for the preparation of compounds of the formula I and salts thereof. It is contemplated that structural elements like N-terminal modified or carboxy-terminal modified derivatives are part of this invention.

The compounds of formula I can have one or more centers of chirality and can therefore occur in various stereoisomeric forms. All such stereoisomeric forms are encompassed by the present invention. Accordingly, the invention relates in particular to the compounds of the formula I in which at least one of the said residues is mentioned as preferred.

Particularly preference is given to the following compounds of the formula I (as used here, the binding molecules B are marked in bold, spacers Q are marked in italics):

| a) | H-Trp-Glu-Tyr-Cys-OH | (P1) |
|---|---|---|
| b) | Ac-Trp-Glu-Tyr-Cys-NH₂ | (P2) |
| c) | H-Trp-Glu-Tyr-homo-Cys-OH | (P3) |
| d) | H-Trp-Glu-Tyr-Gly-Cys-OH | (P4) |
| e) | H-Trp-Glu-Tyr-Aha-Cys-OH | (P5) |
| f) | H-Trp-Glu-Tyr-Gly-Aha-Cys-OH | (P6) |
| g) | H-D-Cys-D-Tyr-D-Glu-D-Trp-OH | (P7) |
| h) | H-Trp-Glu-Phe-Cys-OH | (P8) |
| i) | H-Trp-Glu-1-Nal-Cys-OH | (P9) |
| j) | H-Trp-Glu-Tyr(OMe)-Cys-OH | (P10) |
| k) | H-Trp-Asp-Tyr-Cys-OH | (P11) |
| l) | H-Bpa-Glu-Tyr-Cys-OH | (P12) |
| m) | H-1-Nal-Glu-Tyr-Cys-OH | (P13) |
| n) | H-2-Nal-Glu-Tyr-Cys-OH | (P14) |
| o) | IAA-Glu-Tyr-Cys-OH | (P15) |
| p) | IAA-Asp-Tyr-Cys-OH | (P16) |
| q) | IBA-Glu-Tyr-Cys-OH | (P17) |
| r) | IBA-Asp-Tyr-Cys-OH | (P18) |
| s) | IPA-Glu-Tyr-Cys-OH | (P19) |
| t) | IPA-Asp-Tyr-Cys-OH | (P20) |
| u) | 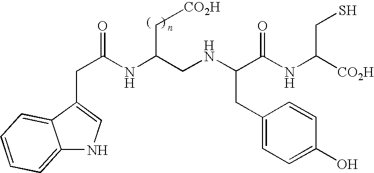 | |
| | n = 1 | (P21) |
| | n = 2 | (P22) |
| v) | 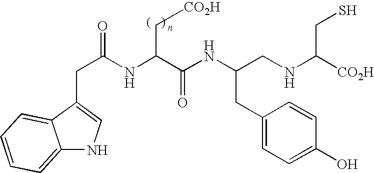 | |
| | n = 1 | (P23) |
| | n = 2 | (P24) |
| w) | 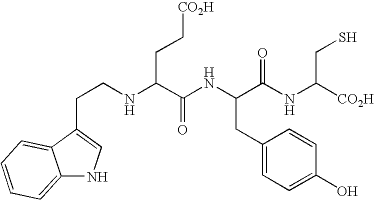 | (P25) |

The compounds of formula I can be understood as non-natural peptides or peptido-mimetic derivatives and may be partially or completely synthesized, for example using solution or solid state synthesis techniques known in the art (Gysin, B. F.; Merrifield, R. B. J. Am. Chem. Soc. 1972, 94, 3102; or Merrifield, R. B. Angew. Chemie Int. Ed. 1985, 24(10), 799-810) applying appropriate amino or carboxy building blocks. A sequential synthesis is contemplated. Other organic synthetic methods may be employed in the synthesis of the compounds according to formula I, such as the methods described in Houben-Weyl, Methods of Organic Chemistry, Georg-Thieme-Verlag, Stuttgart).

If desired, the starting materials can also be formed in situ without isolating them from the reaction mixture, but instead subsequently converting them further into the compounds of the formula I.

Suitable inert solvents are, for example, hydrocarbons, such as hexane, petroleum ether, benzene, toluene, or xylene; chlorinated hydrocarbons, such as trichlorethylene, 1,2-dichloroethane, tetrachloromethane, chloroform or dichloromethane; alcohols, such as methanol, ethanol, isopropanol, n-propanol, n-butanol or tert-butanol; ethers, such as diethyl ether, diisopropyl ether, tetrahydrofurane (THF) or dioxane; glycol ethers, such as 1,2-dimethoxyethane, acetamide, such as N-methylpyrrolidone, dimethylacetamide or dimethylformamide (DMF); nitriles, such as acetonitrile; sulfoxides, such as dimethyl sulfoxide (DMSO); carbon disulfide; carboxylic acids, such as formic acid or acetic acid; nitro compounds, such as nitromethane or nitrobenzene; esters, such as ethyl acetate, water, or mixtures of the said solvents.

The compounds of the formula I can furthermore be obtained by liberation from a functional derivative by solvolysis, such as hydrolysis, or hydrogenolysis.

Preferred starting materials for the solvolysis or hydrogenolysis are those having corresponding protected amino and/or hydroxyl groups instead of one or more free amino and/or hydroxyl groups, preferably those which carry an amino-protecting group instead of an H atom bonded to an N atom, for example those which conform to the formula I, but contain an NHR′ group (in which R′ is an amino-protecting group, for example BOC or CBZ) instead of an NH₂ group.

Preference is furthermore given to starting materials which carry a hydroxyl-protecting group instead of the H atom of a hydroxyl group, for example those which conform to the formula I, but contain an R″Q-phenyl group (in which R″ is a hydroxyl-protecting group, for example tert-butyl or benzyl) instead of a hydroxy-phenyl group. In other words, the hydroxyl group covalently bonded to the aromatic ring is protected from transformation by a protecting group.

Preference is furthermore given to starting materials which carry a carboxyl-protecting group instead of the H atom of a carboxyl group, for example those which conform to the formula I, but contain an R′″O—CO group (in which R′″ is a carboxyl-protecting group, for example tert-butyl or benzyl) instead of a carboxyl group. In other words, the oxygen atom of the carboxyl group is protected from transformation by a protecting group.

It is also possible for more than one—identical or different—protecting group to be present in the molecule. If the more than one protecting groups present are different from one another, this offers an advantage in that they can be cleaved off selectively.

The term “amino-protecting group” is known in general terms and relates to groups which are suitable for protecting (blocking) an amino group against chemical reactions, but are easy to remove. Typical for such groups are, in particular, unsubstituted or substituted acyl, aryl, aralkoxymethyl or aralkyl groups. Because the amino-protecting groups are removed after the desired reaction (or reaction sequence) occurs, their type and size are furthermore not crucial; however, preference is given to those having 1-20, in particular 1-8, carbon atoms. The term “acyl group” includes acyl groups derived from aliphatic, araliphatic, aromatic or heterocyclic carboxylic acids or sulfonic acids, and, in particular, alkoxycarbonyl, aryloxycarbonyl and especially aralkoxycarbonyl groups. Examples of such acyl groups are alkanoyl, such as acetyl, propionyl, butyryl; aralkanoyl such as phenylacetyl; aroyl such as benzoyl oder toluoyl; aryloxyalkanoyl such as POA; alkoxycarbonyl such as methoxycarbonyl, ethoxycarbonyl, 2,2,2-trichlorethoxycarbonyl, BOC, 2-iodethoxycarbonyl; aralkyloxycarbonyl such as CBZ (“carbobenzoxy”), 4-methoxybenzyloxycarbonyl, Fmoc; arylsulfonyl such as Mtr, Pbf or Pmc. Preferred amino-protecting groups are BOC, Mtr, CBZ, Fmoc, Benzyl and Acetyl groups.

The term “hydroxyl-protecting group” is likewise known in general terms and relates to groups which are suitable for protecting a hydroxyl group against chemical reactions, but are easy to remove after the desired chemical reaction has been carried out elsewhere in the molecule. Typical for such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of hydroxyl-protecting groups are, inter alia, benzyl, p-nitrobenzoyl, tert-butyl and acetyl, where benzyl and tert-butyl are particularly preferred.

The term “carboxyl-protecting group” is likewise known in general terms and relates to groups which are suitable for protecting a carboxyl group against chemical reactions, but are easy to remove after the desired chemical reaction has been carried out elsewhere in the molecule. Typical for such groups are the above-mentioned unsubstituted or substituted aryl, aralkyl or acyl groups, furthermore also alkyl groups. The nature and size of the hydroxyl-protecting groups are not crucial since they are removed again after the desired chemical reaction or reaction sequence; preference is given to groups having 1-20, in particular 1-10, carbon atoms. Examples of carboxyl-protecting groups are, inter alia, benzyl, tert-butyl and acetyl, where benzyl and tert-butyl are particularly preferred.

The compounds of the formula I are liberated from their functional derivatives—depending on the protecting group used—for example using strong acids, advantageously using TFA or perchloric acid, but also strong inorganic acids, such as hydrochloric acid or sulfuric acid, strong organic carboxylic acids, such as trichloroacetic acid, or sulfonic acids, such as benzenesulfonic acid or p-toluenesulfonic acid. The presence of an additional inert solvent is possible, but is not always necessary. Suitable inert solvents are preferably organic, for example carboxylic acids, such as acetic acid, ethers, such as tetrahydrofurane or dioxane, amides, such as DMF, halogenated hydrocarbons, such as dichloromethane, furthermore also alcohols, such as methanol, ethanol or isopropanol, and water. Mixtures of above-mentioned solvents are furthermore suitable. TFA is preferably used in excess without addition of a further solvent, and perchloric acid is preferably used in the form of a mixture of acetic acid and 70% perchloric acid in the ratio 9:1. The reaction temperatures for the cleavage are advantageously between about 0° C. and about 50° C., preferably between 15° C. and 30° C. (room temperature).

The BOC, OBut, Pbf, Pmc and Mtr groups can, for example, preferably be cleaved off using TFA in dichloromethane or using approximately 3 to 5N HCl in dioxane at 15-30° C., and the Fmoc group can be cleaved off using an approximately 5 to 50% solution of dimethylamine, diethylamine or piperidine in DMF at 15-30° C.

The trityl group is employed to protect the amino acids histidine, asparagine, glutamine and cysteine. They are cleaved off using TFA/10% thiophenol, TFA/anisole, TFA/thioanisole or TFA/TIPS/H₂O, with the trityl group being cleaved off all the said amino acids.

The Pbf (pentamethylbenzofuranyl) group is employed to protect Arg. It is cleaved off using, for example TFA in dichloromethane.

Hydrogenolytically removable protecting groups (for example CBZ or benzyl) can be cleaved off, for example, by treatment with hydrogen in the presence of a catalyst (for example a noble-metal catalyst, such as palladium, advantageously on a support, such as carbon). Suitable solvents here are those indicated above, in particular, for example, alcohols, such as methanol or ethanol, or amides, such as DMF. The hydrogenolysis is generally carried out at temperatures between 0° C. and 100° C. and pressures between 1 and 200 bar, preferably at 10-30° C. and 1-10 bar. Hydrogenolysis of the CBZ group succeeds well, for example, on 5 to 10% Pd/C in methanol or using ammonium formate (instead of hydrogen) on Pd/C in methanol/DMF at 10-30° C.

A base of the formula I can be converted into the associated acid-addition salt using an acid, for example by reaction of equivalent amounts of the base and the acid in an inert solvent, such as ethanol, followed by evaporation. Thus it is possible to use inorganic acids, for example sulfuric acid, nitric acid, a hydrohalic acid, such as hydrochloric acid or hydrobromic acid, phosphoric acids, such as orthophosphoric acid or sulfamic acid. Organic acids may be employed including aliphatic, alicyclic, araliphatic, aromatic or heteroaromatic monobasic or polybasic carboxylic, sulfonic or sulfuric acids, for example formic acid, acetic acid, trifluoroacetic acid, propionic acid, pivalic acid, diethylacetic acid, malonic acid, succinic acid, pimelic acid, fumaric acid, maleic acid, lacitic acid, tartaric acid, malic acid, citric acid, gluconic acid, ascorbic acid, nicotinic acid, isonicotinic acid, methane- or ethanesulfonic acid, ethanedisulfonic acid, 2-hydroxyethanesulfonic acid, benzenesulfonic acid, p-toluenesulfonic acid, naphthalenemono- and -disulfonic acids and laurylsulfuric acid. Salts, for example picrates, can also be used for the isolation and/or purification of the compounds of the formula I.

On the other hand, an acid of the formula I can be converted into one of its metal of ammonium salts by reaction with a base. Suitable salts here are, in particular, the sodium, potassium, magnesium, calcium and ammonium salts, furthermore substituted ammonium salts, for example the dimethyl-, diethyl- or diisopropyl-ammonium salts, monoethanol-diethanol- or diisopropanolylammonium salts, cyclohexyl-, dicyclohexylammonium salts, dibenzylethylenediammonium salts, furthermore, for example, salts with arginine or lysine.

According to one embodiment of the present invention, an advantageous process for preparing the above-mentioned peptido-mimetics having a reduced peptide bond between Z2 and Z3 is provided:

To obtain larger quantities of such peptides and peptido-mimetics, it is generally considered to be favourable to carry out the synthesis in solution rather than using a solid state synthetic process. However, contrary to a solid state synthetic process, a synthesis in solution includes some reaction steps under mild acidic condition, which may potentially lead to an inadvertent cleaving of acid labile protecting groups such as the trityl protecting group (Zervas, L.; Photaki, I. On Cysteine and Cystine Peptides. 1. New S-Protecting Groups for Cysteine. Journal of the American Chemical Society 1962, 84, 3887-3897).

This problem is particularly pronounced for many of the compounds of the present invention for the following reason. Many compounds of the present invention contain a cysteine residue at the C-terminus because the thiol group of the cysteine can be used for binding the compound to a resin support. However, solution state synthesis is normally carried out from the C-terminus to the N-terminus to thereby suppress problems with racemization side reactions. If this is done with the compounds of the present invention, the cysteine residue is introduced at a very early stage of the multi-step reaction sequence. This, in turn, implies that the trityl protective group of the cysteine residue must withstand a relatively large number of reaction steps. Evidently, this aggravates said trityl-related stability problem.

According to this embodiment of the present invention, a synthetic process is provided that permits to minimize said trityl-related stability problem whilst keeping the risk of racemization side reactions low. That is, it has now been found that trityl-related stability problems can be effectively avoided if the direction of the synthesis is reversed such that the cysteine residue is incorporated into the molecule at the end of the multi-step reaction sequence. It has furthermore been found that, contrary to the synthesis of peptides, no racemization problems arise if the bond between Z2 and Z3 does not contain a carbonyl group. Thus, the present invention provides a method for preparing peptido-mimetics according to the present invention, which are characterized by the absence of a carbonyl group in the bond between residues Z2 and Z3, and which preferably comprise a cysteine residue as the anchoring molecule, and wherein said method is carried out in solution such that the cysteine residue is the last residue to be incorporated.

For illustration purposes, a general description of the synthesis of peptido-mimetic P22 according to this embodiment of the invention is provided below:

Solution phase synthesis of P22. The synthesis starts from 3-indolylacetic acid (35) in which the indole nitrogen is protected with a tert-butyloxycarbonyl (Boc) group according to literature procedures (Scheme 1): Compound 35 is transformed into its methyl ester by treating with SOCl₂ in MeOH and the indole nitrogen is subsequently protected with a Boc group by reacting with tert-butyl dicarbonate and DMAP in acetonitrile to achieve 36. Saponification then yields the desired indolylacetic acid 37.

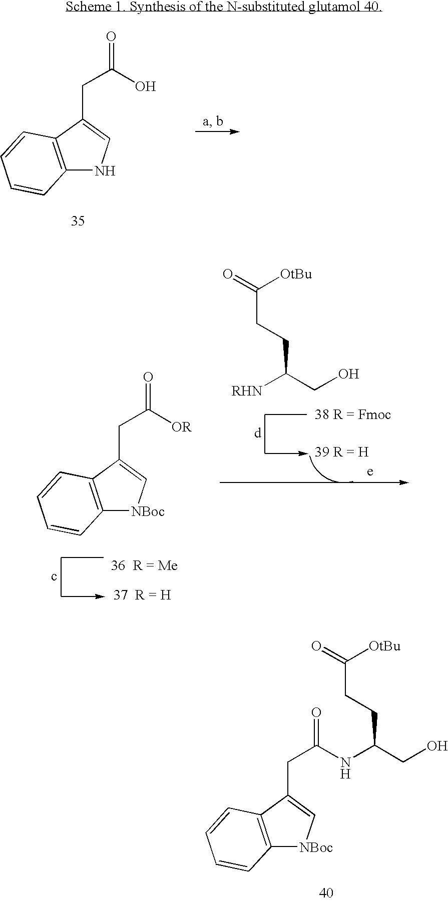

Reagents and conditions: (a) SOCl₂, MeOH, 18 h; (b) Boc₂O, DMAP, acetonitrile, 4 h, (two steps); (c) LiOH, THF, MeOH, H₂O, 18 h; (d) piperidine, DMF, 1 h; (e) HOBt, TBTU, DIPEA, 0° C.→rt, 4 h, (two steps).

The conversion to 40 can be achieved by coupling 37 to the side chain protected glutamol 39 using HOBt and TBTU as coupling reagents. 39 is readily available from commercial Fmoc-Glutamol(OtBu) (37) by treating with piperidin and can be used without further purification. A protection of the free hydroxyl functionality in 39 is not necessary and the reaction proceeds cleanly to give the N-substituted glutamol 40. The reduced peptide bond linking the glutamic acid- and tyrosine residue in the target compound P22 is formed by a reductive amination of the corresponding aldehyde 41 and commercial Tyr(tBu)OMe (Scheme 2).

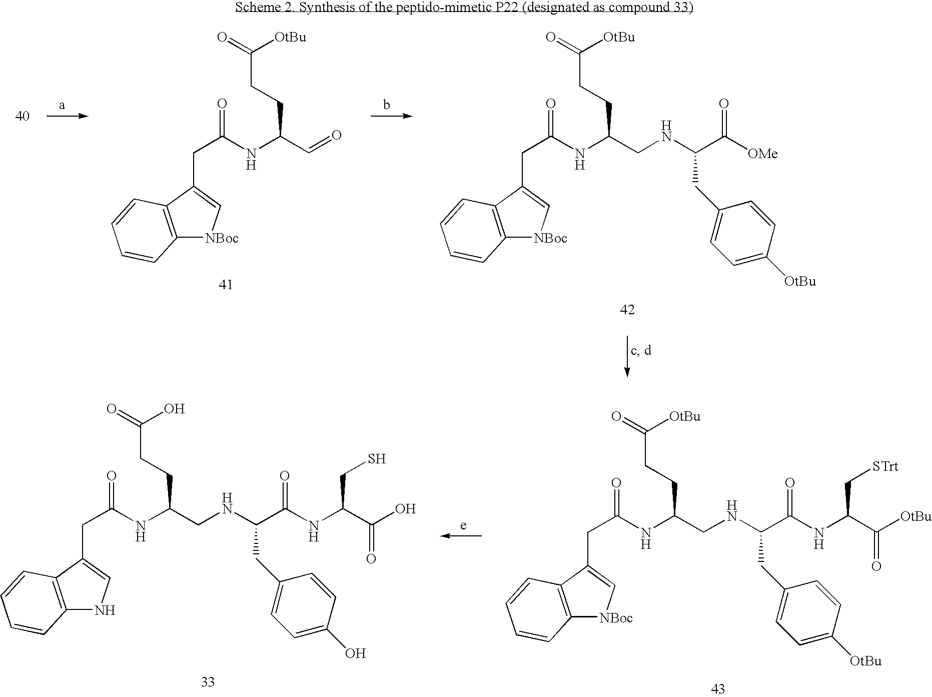

Reagents and conditions: (a) Dess-Martin periodinane, DCM, 6 h; (b) 1) Tyr(tBu)OMe*HCl, MgSO₄, DCM, 30 min; 2) NaB(OAc)₃H, 18 h, (three steps); (c) LiOH, THF, dioxane, H₂O, 1.5 h; (d) Cys(Trt)OtBu*HCl, HOBt, TBTU, 2,4,6-collidine, 10° C.→rt, 18 h, (two steps); (e) TIPS, H₂O, TFA 0 →95%, 8 h.

It is important to avoid basic reaction conditions during the aldehyde formation as this may lead to racemization. Thus, racemization can be completely avoided by the use of Dess-Martin periodinane oxidation and short preformation of the imine in-situ in absence of base, rapidly followed by addition of the reducing agent. This procedure gives the desired secondary amine 42 over three steps. Only traces of the twofold alkylated byproduct are be observed by HPLC-MS. The methyl ester in 42 is cleaved by saponification and the resulting free acid is coupled to Cys(Trt)OtBu*HCl in the presence of HOBt/TBTU and the mild base 2,4,6-collidine to yield 43 (two steps). Although the applied cysteine tert-butyl ester is not commercially available, it is favored over the commercial methyl ester as it is easily synthesizable and it allows a one-step deprotection of 43 to the desired free peptide-mimetic P22 under acidic conditions. In addition, this permits to avoid a significant loss of optical purity after saponification.

The final deprotection and purification is the critical step in terms of an economic production of P22. These steps can be carried out without production of byproducts by suspending P22 in a vigorously stirred mixture of water and TIPS (1:1) and slowly adding the TFA over a period of 8 hours to a final concentration of 95%. By this procedure the byproduct formation is greatly reduced to obtain the final free peptido-mimetic P22 in high yield and high purity after precipitation in ether/pentane.

The compounds of the present invention can be used as described below.

Diagnostic applications: A definite diagnosis for hemophilia A is evaluated by performing a FVIII assay and measuring the clotting time. Therefore, the patient's plasma is mixed with FVIII-deficient plasma from a patient who congenitally lacks FVIII or from an artificially depleted source. The degree of effectiveness in shortening the clotting time will be compared with that of normal plasma. A standard curve is generated using dilutions of pooled fresh normal human plasma with the hemophilic plasma and plotting the clotting times against the dilutions.

Though clotting tests are still the most often performed assays used in preoperative medical screening and for therapy monitoring, these tests rely all on enzymatic steps. Coagulation factors in plasma are usually inactive and require as the first step a proteolytic activation. In addition, these enzymatic steps need not only the activated coagulation factors, but also an activated cofactor, phospholipids and calcium ions. This means that a very complex mixture of relatively unstable proteins is involved in the assay which might underestimate the actual FVIII level. In order to overcome this problem, it is important to additionally evaluate the absolute levels of FVIII in the patient. This is usually done by means of ELISA tests using labile anti-FVIII antibodies. These antibodies can be replaced by the peptides and peptidomimetics of the present invention. When used for this purpose the peptides according to the present invention have major advantages compared to the currently used labile anti-FVIII antibodies employed in ELISA tests. The development of sensitive screening kits for the detection of the total FVIII amount in the patient's plasma permits to benefit from the advantages of the peptides which are found in their greater stability, higher sensitivity and lower assay costs.

Use for stabilization purposes: FVIII shows rapid inactivation and a short half-life. The half-life of FVIII is defined by the rate of spontaneous dissociation of the A2 subunit from active heterotrimeric FVIII (A1/A2/A3-C1-C2) in which the A2 subunit is weakly associated with the A1 and the A3-C1-C2 subunits via ionic interactions. The presence of A2 in the heterotrimer is required for normal stability of active FVIII.

The peptides and peptidomimetics of the present invention exhibit not only a high affinity to FVIII, but, upon binding, they also serve to stabilize the heterotrimer. A binding of these inventive compounds to FVIII can therefore be used in an advantageous manner in hemophilia A therapy to thereby increase the stability and half-life of FVIII during medical treatment. A longer half-life of FVIII during substitution therapy will ease the patient's well-being as it permits to lower the FVIII infusion frequency. Said stabilization effect may also be used for advantageously increasing the shelf-life of FVIII-containing medicaments prior to their administration.

Use for labeling, detecting and identifying: The compounds of the present invention may also carry a marker group such as a radioactive isotope or a functional group that can undergo a colour reaction or the like. The contacting of such compounds of the present invention with FVIII will lead to the binding of the marker peptide or peptidomimetics to FVIII. This, in turn, permits to detect and, as the case may be, quantify the FVIII present in a sample. Use in therapy: In addition to the above-mentioned stabilization effect, the compounds of the present invention may furthermore increase the biological activity of FVIII. In addition, the compounds of the present application may have the advantageous effect of inhibiting the binding of antibodies to the administered FVIII. These beneficial effects may be used in therapy by contacting FVIII with a compound of the present invention prior to its administration. The compound of the present invention will bind to FVIII thus forming a complex. Administration of this complex instead of the pure FVIII may lead to an increased biological effect (or, alternatively, permit to administer lower dosages of FVIII). Moreover, this administration of this complex may be helpful in reducing the deactivating effect of antibodies. In short, the compounds of the present invention may be used for manufacturing a FVIII-based medicament that exhibits higher stability and superior activity as compared with conventional FVIII. Said FVIII-based medicament may also be used for substituting conventional FVIII in cases where said conventional FVIII is deactivated by antibodies. Moreover, the present invention also pertains to a method for treating hemophilia A that includes the step of administering an effective dose of said complex of FVIII and the compound of the present invention to a subject in need thereof. Use in the manufacture of FVIII-based medicaments: Another embodiment of the present invention pertains to the use of the compounds of the present invention for purifying raw FVIII and FVIII-like proteins. This involves preferably the immobilization of the compounds of the present invention on a solid support. More preferably, an affinity chromatography is carried out using a resin coated with the compounds of the present invention. Such uses are described in more detail in Examples 3 to 5 below.

Use in the manufacture of diagnostics and/or research tools: The present invention furthermore pertains to the use of the compounds of the present invention for purifying domains, epitopes and fragments of FVIII and FVIII-like proteins. Whilst such purified domains and the like may not exhibit a clotting activity comparable to FVIII, they may nevertheless be useful in diagnostic kits, as research tools and the like.

### EXAMPLES

Chromatographic methods were used according to the following parameters: RT=retention time (minutes) on HPLC in the following system:

### Column: YMC ODS A RP 5C₁₈, 250×4.6 mm

Eluent A: 0.1% TFA in water
Eluent B: 0.1% TFA in acetonitrile
Flow rate: 1 mL/min

### Gradient: 10->50% B/30 min.

Mass spectrometry (MS): ESI (electrospray ionisation) (M+H)⁺

SDS-polyacrylamide gel electrophoresis (PAGE): FVIII SDS-PAGE was performed using 10% Tris-Glycine Bio-Rad ReadyGel. Samples were diluted in loading buffer containing 2-Mercaptoethanol and applied to the gel. Electrophoresis was performed at constant current (25 mA/gel) in Bio-Rad Mini-Protean 3 apparatus put on ice. After electrophoresis gel was stained using a standard silver staining protocol.

Immunoblotting: The proteins were transferred to nitrocellulose membrane in Bio-Rad Mini-Protean 3 apparatus with transfer block. Transfer was performed at constant 70 V voltage (˜250 mA) for 4 hours with frozen cooling block installed. Membrane was blocked overnight with 5% non-fat dry milk in TBS, pH 8.0. Then membrane was incubated for 1 hour with primary antibodies diluted to 1 μg/mL in 5% non-fat dry milk solution in TBS pH=7.4 containing 0.1% Tween-20.

Membrane was washed 3 times in TBS (pH=7.4) containing 0.1% Tween-20 and incubated for 1 hour with peroxidase-labeled goat anti-mouse antibodies (Mab C5 and Mab 413) diluted 1:3000 in 5% non-fat dry milk in TBS pH=7.4, 0.1% Tween-20. After 3 washes with TBS, 0.1% Tween-20 membrane was developed in ECL chemiluminescence substrate (Pharmacia) and chemiluminescence was detected using BioMax-XL film (Kodak).

Rink-amid resin stands for 4-(2′,4′-Dimethoxyphenyl-Fmoc-aminomethyl)-phenoxy resin, which allows, for example, the synthesis of peptides and peptido mimetic derivatives with C-terminal —CONH2 groups, TCP resin denotes trityl chloride-polystyrene resin.

The compounds P1 to P20 were synthesized via solid phase peptide synthesis using Fmoc-strategy on TCP resin and on Rink-amide resin for compound P2, respectively (see Fields, G. B.; Nobie, R. L. Int. J. Pept. Protein Res. 1990, 35, 161).

N-Terminal acetylation (compound P2) was accomplished on solid phase by treating the corresponding N-terminal deprotected compound with a NMP/Ac₂O/DIPEA (91:7:2) mixture prior to the final cleavage and deprotection step (see below).

The compounds P21 to P25 were completely synthesized sequentially on TCP resin using Fmoc-strategy. The “reduced peptide bonds” were formed via a reductive alkylation on solid phase as known per se using an the amino acid corresponding aldehyde (“amino aldehyde”, see Krchnak, V.; Weichsel, A. S.; Cabel, D.; Flegelova, Z.; Lebl, M. Mol. Diversity. 1995, 1, 149). The reaction was carried out in a water trapping solvent like trimethoxymethane at room temperature. The reduction of the corresponding imine compound formed as an intermediate product was performed in an aprotic solvent such as dichloromethane at room temperature. As reducing agents, complex boron hydrides like NaBH(OAc)₃ were used.

The cleavage of the peptides or peptido-mimetic derivatives from the solid phase and the cleavage of their side chain protection groups was done simultaneously using 90% TFA, 5% H₂O and 5% TIPS.

All compounds were purified by preparative HPLC.

### Example 1

### Preparation of Compounds as Affinity Ligands for FVIII and Binding of pd-FVIII

Peptides P1 to P25 were immobilized on the Toyopearl AF-Epoxy-650M resin (Tosoh Biosep) as described by Jungbauer et al. For immobilization, 2.5 mg of each peptide was dissolved in 0.25 mL of the immobilization buffer (0.2 M sodium bicarbonate, pH 10.3), and 0.036 g of the dry resin powder (corresponding to 0.125 mL of swollen resin) was added, followed by incubation of the mixture with gentle rotation for 48 hours. Upon incubation for 48 hours the resin was washed once with immobilization buffer, once with 1 M NaCl and then 3 times with binding buffer, and binding of ¹²⁵I-labeled pd-FVIII to the peptide-coated and control resin was tested. The coupling density of each peptide in each of the reported experiments was as mentioned in Table 1. The control 0.25 mL portion of the resin was similarly treated in parallel experiment in the absence of peptide and was subsequently used as a control (designated as Background) in ¹²⁵I-pd-FVIII binding experiments. ¹²⁵I-pd-FVIII Bound/Background ratios were calculated as the amount of ¹²⁵I-pd-FVIII, bound to an immobilized peptide, divided by that bound to uncoated control resin, prepared as described above. This ratio represents a Signal/Noise ratio for the micro-beads assay, since ¹²⁵I-pd-FVIII bound to peptide represents the signal value and ¹²⁵I-pd-FVIII bound to peptide-uncoated resin represents the background (noise) value.

Plasma-derived (pd-) human Factor VIII (FVIII) was purified from concentrate by immunoaffinity chromatography on an anti-FVIII monoclonal antibody column followed by subsequent concentration of pd-FVIII by ion-exchange chromatography using Resource Q HR5/5 column. To separate FVIII from vWf, concentrate was incubated in 0.35 M NaCl, 0.04 M CaCl₂, prior to affinity purification.

Trace amounts of vWf, which are potentially present in pd-FVIII preparation, were removed by passing pd-FVIII preparation through the column with anti-vWf high affinity monoclonal antibody, immobilized at the density 1.4 mg per mL of resin.

Ten μg of purified pd-FVIII were iodinated using lactoperoxidase beads and 0.5 mCi of Na¹²⁵I.

The resin with immobilized peptides was washed in the binding buffer (0.01 M Hepes, 0.1 M NaCl, 5 mM CaCl₂, 0.01% Tween-80). Subsequently, the resin was diluted in the binding buffer as 1:7 slurry and aliquoted into Eppendorf tubes (40 μl per tube). ¹²⁵I-pd-FVIII (100000 cpm in 10 μl) was added to the tubes and the volume of the mixture was adjusted to 100 μl by adding 50 μl of the binding buffer containing 4% BSA to give a 2% final concentration of BSA. After 2 hours of incubation at room temperature on a rotator, the samples were washed 4 times in the binding. After each wash the tubes were centrifuged at 5000 rpm for 1 min in an Eppendorf microcentrifuge, supernatant was discarded, and the resin was re-suspended in the washing buffer, followed by centrifugation under the same conditions. After four washes the tubes with pellet without supernatant were counted for radioactivity. The resin without peptide was processed similarly to account for non-specific pd-FVIII binding to tubes and resin itself, and the radioactivity in this control tube was considered as a background value. Since ¹²⁵I-pd-FVIII contains the some fraction damaged during radiolabeling, binding was calculated as a percent of maximal achievable binding, determined in separate experiment with anti-FVIII Mab 8860-coated resin.

All the measurements were performed in duplicates. Each experiment was performed using two independently prepared immobilized peptide samples. The data presented in each figure are the mean values of the four determinations: duplicate determination in the two assays performed with beads on which peptides were independently immobilized on the different days. The value of standard deviation of the above quadruplicate determinations (duplicate determinations in two independent experiments) were typically less than 10% of the measured values of ¹²⁵I-pd-FVIII binding to peptides.

TABLE 1
Binding of 125I-labeled Factor VIII to compounds P1 to P10 immobilized
on Toyopearl ® AF-Epoxy-650M.

| Compound | | | % Binding | Binding, rel. to | Loading density |
|---|---|---|---|---|---|
| (Label) | RT (min) | MS (ESI): m/z | pdFVIII | control | in μmol/mL |
| P1 | 16.9 | 600.4 (M + H)⁺ | 47.6 ± 4.0 | 20.9 ± 1.7 | 18.2 |
| | | Calcd: 599.2 | | | |
| P2 | 20.5 | 627.1 (M + H)⁺ | 53.6 ± 5.7 | 18.9 ± 2.0 | 23.0 |
| | | Calcd: 626.2 | | | |
| P3 | 19.9 | 614.4 (M + H)⁺ | 38.4 ± 2.3 | 21.9 ± 1.3 | 12.0 |
| | | Calcd: 613.22 | | | |
| P4 | 18.1 | 657.4 (M + H)⁺ | 52.7 ± 2.5 | 27.4 ± 1.3 | 14.9 |
| | | Calcd: 656.2 | | | |
| P5 | 20.9 | 713.5 (M + H)⁺ | 42.7 ± 2.3 | 22.2 ± 1.9 | 13.6 |
| | | Calcd: 712.3 | | | |
| P6 | 17.9 | 770.3 (M + H)⁺ | 43.1 ± 2.0 | 15.2 ± 0.7 | 10.1 |
| | | Calcd: 769.3 | | | |
| P7 | 20.4 | 600.4 (M + H)⁺ | 56.1 ± 0.9 | 19.8 ± 0.3 | 17.1 |
| | | Calcd: 599.2 | | | |
| P8 | 21.3 | 584.2 (M + H)⁺ | 24.7 ± 0.1 | 8.7 ± 0.1 | 11.5 |
| | | Calcd: 583.2 | | | |
| P9 | 25.5 | 634.2 (M + H)⁺ | 60.9 ± 5.5 | 23.8 ± 2.2 | 19.7 |
| | | Calcd: 633.23 | | | |
| P10 | 21.9 | 614.2 (M + H)⁺ | 54.1 ± 8.4 | 19.0 ± 3.0 | 16.6 |
| | | Calcd: 613.2 | | | |
| P11 | 17.2 | 586.1 (M + H)⁺ | 62.8 ± 6.9 | 24.7 ± 2.7 | 17.6 |
| | | Calcd: 585.2 | | | |
| P12 | 22.0 | 665.2 (M + H)⁺ | 57.0 ± 1.39 | 22.3 ± 0.5 | 20.2 |
| | | Calcd: 664.2 | | | |
| P13 | 21.1 | 611.2 (M + H)⁺ | 66.8 ± 5.9 | 26.1 ± 2.3 | 20.3 |
| | | Calcd: 610.2 | | | |
| P14 | 21.3 | 611.2 (M + H)⁺ | 58.1 ± 6.1 | 22.7 ± 2.4 | 19.9 |
| | | Calcd: 610.2 | | | |
| P15 | 21.1 | 571.3 (M + H)⁺ | 75.4 ± 0.6 | 39.3 ± 0.3 | 19.5 |
| | | Calcd: 570.2 | | | |
| P16 | 21.4 | 557.0 (M + H)⁺ | 70.1 ± 0.8 | 24.7 ± 0.3 | 19.8 |
| | | Calcd: 556.2 | | | |
| P17 | 25.3 | 599.1 (M + H)⁺ | 62.7 ± 3.9 | 24.7 ± 1.5 | 22.2 |
| | | Calcd: 598.2 | | | |
| P18 | 25.0 | 585.1 (M + H)⁺ | 57.2 ± 7.2 | 20.2 ± 2.5 | 21.2 |
| | | Calcd: 584.2 | | | |
| P19 | 23.2 | 585.1 (M + H)⁺ | 53.2 ± 4.9 | 18.7 ± 1.7 | 26.8 |
| | | Calcd: 584.2 | | | |
| P20 | 23.0 | 571.3 (M + H)⁺ | 60.7 ± 4.1 | 21.4 ± 1.4 | 23.1 |
| | | Calcd: 570.2 | | | |
| P21 | 18.2 | 543.2 (M + H)⁺ | 47.8 ± 2.7 | 27.3 ± 1.6 | 18.2 |
| | | Calcd: 542.6 | | | |
| P22 | 18.5 | 557.5 (M + H)⁺ | 65.3 ± 3.0 | 34.5 ± 0.2 | 17.8 |
| | | Calcd: 556.6 | | | |
| P23 | 18.2 | 543.3 (M + H)⁺ | 38.5 ± 5.6 | 22.0 ± 3.2 | 13.9 |
| | | Calcd: 542.6 | | | |
| P24 | 18.4 | 557.3 (M + H)⁺ | 43.4 ± 2.3 | 24.8 ± 1.3 | 13.7 |
| | | Calcd: 556.6 | | | |
| P25 | 19.5 | 557.4 (M + H)⁺ | 53.7 ± 1.9 | 22.7 ± 0.8 | 18.4 |
| | | Calcd: 556.6 | | | |

### Comparative Example 1

### Preparation of a Comparative Compound with Scrabled Sequence as a Comparative Ligand for FVIII and Binding of pd-FVIII

The procedure described in Example 1 was repeated using a peptide with an arbitrary scrambled amino acid sequence (ECYYEHWS). Subsequently, the FVIII binding to the resin carrying this scrambled peptide as well as FVIII binding to the uncoated resin were investigated in the same manner as described above with respect to Example 1. The results are shown in the following Table 2.

TABLE 2
Binding of 125I-labeled Factor VIII to a comparative compound
immobilized on Toyopearl ® AF-Epoxy-650M and to
the same resin in uncoated form.

| | | | | Binding, | Loading |
|---|---|---|---|---|---|
| | RT | MS (ESI): | % Binding | rel. to | density in |
| Compound | (min) | m/z | pdFVIII | control | μmol/mL |
| scrambled^{a} | 16.2 | 1116.6 | 6.5 ± 0.5 | 4.1 ± 0.3 | 10.3 ± 0.4 |
| | | (M + H)⁺ | | | |
| | | calcd: | | | |
| | | 1115.4 | | | |
| control^{b} | — | — | 1.6 ± 0.5 | — | — |

^{a}Sequence: ECYYEHWS;
^{b}uncoated resin

A comparison of the results reported in Example 1 with those of Comparative Example 1 shows that the compounds of the present invention exhibit a significantly higher affinity to FVIII as compared with the scrambled peptide.

### Example 2

### Binding of Recombinant FVIII Using P15 Coated Resin and P22 Coated Resin

Kogenate® and ReFacto® are recombinant forms of FVIII that are commercially available from Bayer as well as Wyeth-Ayerst Pharmacia and Upjohn, respectively.

Kogenate® was purified from total amount of 4000 IU (5 vials) using immune affinity chromatography followed by ion-exchange chromatography using Resource Q HR5/5 column with a linear gradient of NaCl. Purified Kogenate® had a concentration of 130 μg/ml, activity of 740 IU/mL, and specific activity of 5700 IU/μg. ReFacto® was purified from total amount of 5000 IU (5 vials) using immune affinity chromatography followed by ion-exchange chromatography using Resource Q HR5/5 column. Purified ReFacto® had a concentration of 89 μg/mL, activity of 864 IU/mL, and specific activity of 9707 IU/μg.

Kogenate® and ReFacto® were iodinated in the same manner as described in Example 1 with respect to the pd-FVIII. Protein binding was measured using the same procedures as described in Example 1 above. The results of this experiment are summarized in the following Table 3.

TABLE 3
Binding of recombinant FVIII to resin coated with P15 and resin
coated with P22

| | | ¹²⁵I-ReFacto | ¹²⁵I-Kogenated |
|---|---|---|---|
| | Peptide density | binding (% of | FS binding (% of |
| Sequence no. | (μmol/mL) | total) | total) |
| P15 | 19.2 ± 1.0 | 54.9 ± 6.3 | 49.6 ± 1.7 |
| P22 | 17.3 ± 0.9 | 53.3 ± 2.3 | 40.6 ± 2.3 |

The results of this experiment demonstrate that the compounds of the present invention exhibit also a high affinity with respect to FVIII-like proteins such as recombinant FVIII.

### Example 3

### Purification of Active pd-FVIII Using P22 Coated Resin

The peptido-mimetic derivative P22 was immobilized on the Toyopearl resin as described in Example 1. 25 mg of peptide and 360 mg of resin were used. The resulting resin (˜1 ml) was packed in a glass column (Pharmacia-Biotech). The purification procedure was performed using a Waters 650E Advanced Protein Purification System. Buffer A was 0.01 M Hepes, 0.1 M NaCl, 5 mM CaCl₂, 0.01% Tween-80 and Buffer B was 0.01 M Hepes, 1 M NaCl, 5 mM CaCl₂, 0.01% Tween-80 (pH 6.8). The elution was monitored by a flow-through UV detector (Waters 490 E) by optical density at 280 nm (OD280). The elution fractions were then analyzed for their protein content by determining OD280 and FVIII activity was determined in a one-stage APTT assay using MLA Electra-800 automatic coagulation timer. The samples from elution fractions were analyzed by 10% PAGE followed by silver staining and Western blotting using monoclonal antibodies against FVIII.

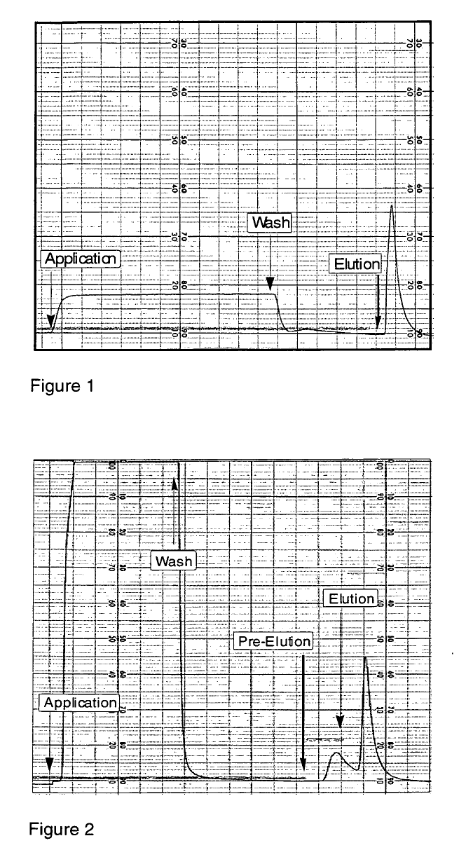

FVIII (0.5 mg), previously purified by immunoaffinity and ion-exchange chromatography as described above, was diluted by 0.01 M Hepes, 5 mM CaCl₂, 0.01% Tween-80 to a final salt concentration of 0.1 M NaCl. The mixture was applied onto the P22-column, followed by wash with Buffer A, until the OD280 returned to background. The bound protein was eluted by 20% Buffer A 80% Buffer B. The elution profile is shown in FIG. 1.

The purification of FVIII was performed from cell-conditioned FBS-containing SF9 media, spiked with FVIII. FVIII (0.5 mg), previously purified by immunoaffinity and ion-exchange chromatography as described above, was mixed with cell-conditioned FBS-containing SF9 media, which was diluted with 0.01 M Hepes, 5 mM CaCl₂, 0.01% Tween-80 to a final salt concentration of 0.1 M NaCl. The mixture was applied onto the column, followed by wash with Buffer A, until the OD280 returned to background. The wash with 85% Buffer A 15% Buffer B was performed to elute some of bound contaminating proteins. The bound protein was eluted by 40% Buffer A 60% Buffer B. The elution profile is shown in FIG. 2.

TABLE 4
Quantitative parameters of FVIII purification from FBS-containing
media.

| | | | Flow- | | Column | Purification, |
|---|---|---|---|---|---|---|
| Material | | Source | through | Elution | retention | fold |
| FBS- | OD 280 | 0.64 | 0.47 | 0.056 | 88.5% | 63 |
| containing | Activity, IU/ml | 29.03 | 3.36 | 160 | | |
| conditioned | | | | | | |
| DMEM | | | | | | |

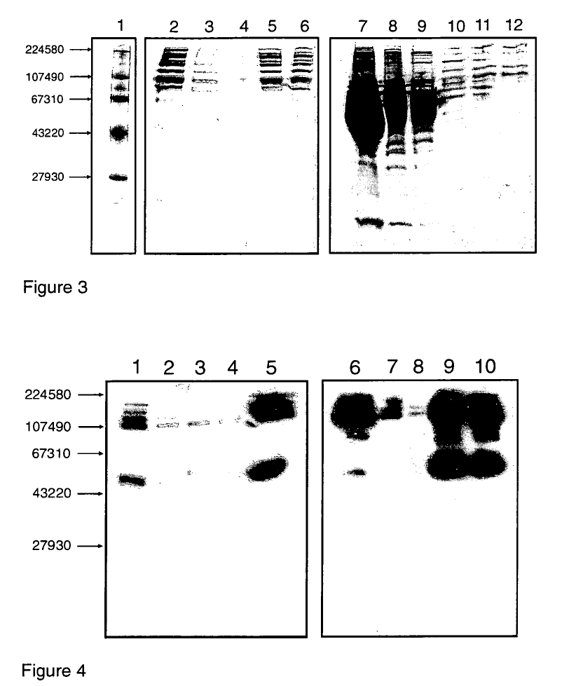

In both purifications, high-satisfactory column retention and successful elution were achieved (FIGS. 3 and 4). During the purification of FVIII from FBS-containing media the contaminant proteins, presented in vast excess to FVIII in source solutions were successfully removed (FIGS. 3 and 4, Table 4).

The peptidomimetic-purified FVIII samples were visually distinguished from the following bands, taking a commercially available pure FVIII preparation as the positive control (lane 2, FIG. 3, lane 2 FIG. 4): 230-90 kDa heavy chain bands, heterogeneous due to different proteolysis of B-domain, and ˜80 kDa light chain doublet bands (due to different glycosilation), which are often irresolvable, single proteolytic band with molecular weight of ˜55 kDa, and another proteolytic band with molecular weight of ˜45 kDa. Neither preparation contained any detectable quantities of some deeper proteolysis of the 45 kDa heavy chain-derived proteolytic band. SDS-PAGE demonstrated that the elution fractions from the purification from previously purified FVIII and from the purification from FBS-containing DMEM conditioned medium, spiked with FVIII have basically the same number of protein bands and the distribution of the material between the bands did not substantially differ from the positive control FVIII. These results confirm that the peptide-purified FVIII preparations from either cell-conditioned or pure background showed the same SDS-PAGE and Western blot pattern than the commercially immunoaffinity purified FVIII control.

### Example 4

### Purification of FVIII with P1 Coated Resin

Peptide P1 was bonded to the resin as described in Example 1. 25 mg of peptide and 360 mg of resin were used. The resulting resin (˜1 ml) was packed in a glass column (Pharmacia-Biotech). A FVIII containing sample was purified as described with respect to Example 3, the only difference between the two experiments being the absence of a prelusion step with Buffer A in the present experiment.

The details of the purification are summarized in the following Table 5:

| | | | Flow- | | Purification, |
|---|---|---|---|---|---|
| Material | | Source | through | Elution | times |
| Pure fVIII | OD 280 | 0.087 | 0.02 | 0.108 | 2 |
| | Activity, | 222 | 9 | 552 | |
| | IU/ml | | | | |
| FBS- | OD 280 | 0.52 | 0.49 | 0.08 | 13 |
| containing | Activity, | 33 | 6 | 66 | |
| conditioned | IU/ml | | | | |
| DMEM | | | | | |

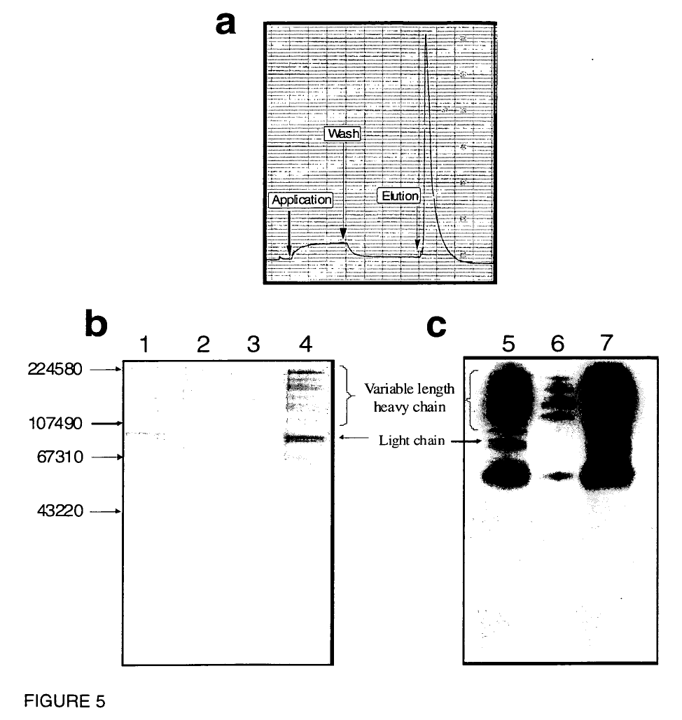

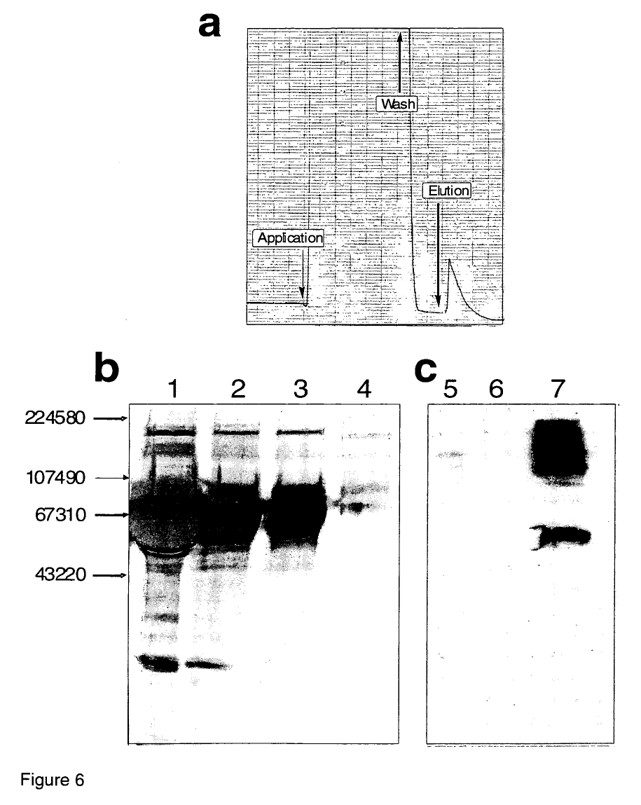

In both purifications, high-satisfactory column retention and successful elution were achieved (FIGS. 5 and 6). During the purification of FVIII from FBS-containing media the contaminant proteins, presented in vast excess to FVIII in source solutions were successfully removed (FIG. 6, Table 5).

1. Compound of the formula I
B-Q-X (I)
wherein B represents Z1-Z2-Z3,
wherein Z1 is a natural occurring or non-proteinogenic amino acid residue or a derivative thereof with a monocyclic, bicyclic or tricyclic side chain having 6 to 25 carbon atoms wherein one or more of these carbon atoms may be replaced by a heteroatom selected from N, O and S, or wherein
Z1 is a residue of the formula
Ar—(CH₂)ₘ—(CHR¹)ₙ—(CH₂)ₒ-A¹ (II)
wherein
A¹ represents a group selected from NR², CO, OCO, CHR², O or S,
R¹ represents a group selected from C₁₋₄ alkyl, phenyl, benzyl, and N(R²)₂, wherein the alkyl, phenyl or benzyl group may carry one or more substituents independently selected from A and N(R²)₂, wherein two or more A's and/or two or more R²'s may be the same or different from each other,
Ar is an aromatic group having a mono-, bi- or tricyclic aromatic ring system with 6 to 14 carbon atoms, a saturated or partially unsaturated C5-14 mono- or bicyclic alkyl group, each of which may be unsubstituted or carry one to three substituents independently selected from A, Ar¹, O—Ar¹, C(O)—Ar¹, CH₂—Ar¹, OH, OA, CF₃, OCF₃, CN, NO₂, Hal; or Ar may be Het,
Hal is selected from F, Cl, Br or I,
Ar¹ is an aromatic group having a mono-, bi- or tricyclic aromatic ring system with 6 to 14 carbon atoms, preferably a phenyl group or a naphthyl group, more preferably a phenyl group. Ar¹ may itself be unsubstituted or carry one to three substituents independently selected from A, OH, OA, CF₃, OCF₃, CN, NO₂, and Hal;
Het represents an optionally substituted saturated, partially or completely unsaturated mono- or bicyclic heterocyclic residue with 5 to 12 ring members, comprising 1 to 3 N- and/or 1 S- or O-atoms
A represents COOR², N(R²)₂ or a linear, branched or cyclic alkyl group with 1-6 C-atoms, which may be unsubstituted or be substituted with COOR² or N(R²)₂,
m and o are independently selected from 0, 1, 2, 3 and 4,
n is 0 or 1, and
R² is H, C₁₋₄ alkyl, phenyl or benzyl or, in the case of peptoid-amino acids, the amino acid side chain;
Z2 represents a naturally occurring or non-natural amino acid, which does not carry an aromatic group in its side-chain;
Z3 represents a group that is as defined above with respect to Z1, wherein Z1 and Z3 may be the same or different from each other, with the proviso that the different positions of Z1 and Z3 within the compound are accounted for by the absence or presence of additional hydrogen atoms in the positions of bonded or free valencies, respectively;
Q is absent or is an organic spacer molecule with 1 to 100 backbone atoms;
X is absent or is an organic anchoring molecule with a terminal group selected from SH, N₃, NH—NH₂, O—NH₂, NH₂, Cl, Br, I, C≡CH, CR⁵O, or carboxyl, wherein
R⁵ is selected from H, C₁₋₄ alkyl or a phenyl or benzyl group that may be unsubstituted or one-, two-, or threefold independently substituted with A, OH, OA, CF₃, OCF₃, CN, NO₂ or Hal,
as well as salts thereof.

2. Compound of the formula I
B-Q-X (I)
comprising
B which is a dipeptide, tripeptide or peptido-mimetic providing affinity to FVIII and/or FVIII-like proteins,
Q which is missing or is an organic spacer molecule and
X which is missing or is an organic anchoring molecule,
as well as their salts.

3. Compound of claim 2 wherein
B is Z1-Z2-Z3
comprising
Z1 a natural occurring or non-proteinogenic amino acid residue or a derivative thereof or Ar—(CH₂)ₘ—(CHR¹)ₙ—(CH₂)ₒ-A¹,
Z2 which is missing or a natural occurring or non-proteinogenic amino acid residue or a derivative thereof,
Z3 a natural occurring or non-proteinogenic amino acid residue or a derivative thereof or Ar—(CH₂)ₙ—(CHR¹)ₘ—(CH₂)ₒ-A¹,
A¹ NR², CO, CHR², O or S,
R¹ C1-4 alkyl, phenyl or benzyl,
Ar unsubstituted or with A, OH, OA, CF₃, OCF₃, CN, NO₂ or Hal one-, two-, or threefold substituted phenyl, which can be substituted one-, two-, or threefold with an A, OH, OA, CF₃, OCF₃, CN, NO₂ or Hal substituted phenyl in such a way that an unsubstituted or substituted biphenyl is created, or Het,
Hal F, Cl, Br or I,
Het a saturated, partially or completely unsaturated mono- or bicyclic heterocyclic residue with 5 to 12 ring members, comprising 1 to 3 N- and/or 1 S- or O-atoms, where the heterocyclic residue can be substituted one- or two-fold with CN, Hal, OH, NH₂, COOH, OA, CF₃, A, NO₂, Ar or OCF₃,
A COOH, NH₂ or alkyl with 1-6 C-atoms, unsubstituted or substituted with COOH or NH₂,
m, o 0, 1, 2, 3 or 4,
n 0 or 1,
whereby
Z1 and Z3 or Z1 and Z2 as well as Z2 and Z3 can be bound through an acid-amide bond —CO—NR²— or —NR²—CO—, preferentially through a so called reduced peptide bond —CH₂—NR²— or —NR²—CH₂— as well as through —CO—CHR²—, —CHR²—CO—, —CR²═CH— or —CH═CR²— bonds
wherein
R² is H, C1-4 alkyl, phenyl or benzyl or, in the case of peptoid-amino acids, the amino acid side chain.

4. Compound of claim 2 or 3, wherein Q is an organic spacer molecule selected from one of the following groups
[—NH—(CH₂)ₓ—CO]_{w},
[—NH—(CH₂CH₂—O—)_{y}CH₂—CO]_{w},
[CO—(CH₂)_{z}—CO—],
[NH—(CH₂)_{z}—NH—],
[CO—CH₂—(OCH₂CH₂)_{y}—O—CH₂—CO—],
[NH—CH₂CH₂—(OCH₂CH₂)_{y}—NH—],
amino acids, peptides, saccharides or polyethers as well as their combinations
wherein
w is independently from 1 to 8 respectively
x is 1-5
y is 1-6 and
z is 1-6.

5. Compound of claim 2, 3 or 4 wherein X is an organic anchoring molecule selected from one of the following groups
a naturally occurring or non-proteinogenic amino acid,
-A¹-(CH₂)ₚ-A²,
-A¹-CH₂—(OCH₂CH₂)_{y}—O—CH₂-A²,
-A1-CH₂CH₂—(OCH₂CH₂)_{y}-A²,
═CR²—(CH₂)ₚ-A²,
-A¹-CH(NHR³)—(CH₂)_{q}-A²,
═CR²—CH(NHR³)—(CH₂)_{q}-A²,
-A¹-CH(COR⁴)—(CH₂)_{q}-A² or
═CR²—CH(COR⁴)—(CH₂)_{q}-A²,
wherein
A¹ is NR², CO, CHR², O, or S,
A² is SH, N₃, NH—NH₂, O—NH₂, NH₂, Hal¹, C≡CH, CR⁵O, or carboxyl,
R² is as defined in claim 2,
R³ is H, R⁶, —COR⁶, —COOR⁶,
R⁴ is —OR⁶ or —NHR³,
R⁵ is H, C1-4 alkyl or unsubstituted or with A, OH, OA, CF₃, OCF₃, CN, NO₂ or Hal one-, two-, or threefold substituted phenyl or benzyl,
R⁶ is H, C1-4 alkyl or unsubstituted or with A, OH, OA, CF₃, OCF₃, CN, NO₂ or Hal one-, two-, or threefold substituted phenyl or benzyl,
p is 1-20,
q is 1-20,
y is 1-6,
z is 1-6,
Hal is as defined in claim 3,
Hal¹ is Cl, Br or I.

6. Compound according to any one of the preceding claims 2 to 5 further characterized that
Z1 means Ar—(CH₂)ₘ—(CHR³)ₙ—(CH₂)ₒ—CO—
whereby
Ar means 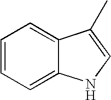
and m, n and o are as defined in claim 3.

7. Compound according to any one of the preceding claims 2 to 6, further characterized that
m means 1, 2 or 3, n means 0 and o means 0.

8. Compound according to any one of the claims 2 to 5, further characterized that
Z1 means 1-Nal, 2-Nal, Bpa or R⁷-Trp
whereby
R⁷ is H, R⁸, —COR⁸, —COOR⁸ wherein
R⁸ is C1-4 alkyl or unsubstituted or with A, OH, OA, CF₃, OCF₃, CN, NO₂ or Hal one-, two-, or threefold substituted phenyl or benzyl
and A and Hal are as defined in claim 3.

9. Compound according to any one of the preceding claims 2 to 8, further characterized that
R⁷ means H or —COR⁸
wherein
R⁸ means methyl.

10. Compound according to any one of the preceding claims 2 to 9, further characterized that
Z2 is an amino acid residue with a glutamic or aspartic side chain.

11. Compound according to any one of the preceding claims 2 to 10, further characterized that
Z3 is an amino acid residue with a 1-naphthylalanine, phenylalanine, tyrosine or O-methylated tyrosine side chain.

12. Compound according to any one of the preceding claims 2 to 11, further characterized that
Q means [—NH—(CH₂)ₓ—CO]_{w}
and x and w are as defined in claim 4.

13. Compound according to any one of the preceding claims 2 to 12, further characterized that
x means 1 or 5 and w means 0 or 1.

14. Compound according to any one of the preceding claims 2 to 13, further characterized that
X means -A¹-CH(COR⁴)—(CH₂)_{q}-A²
wherein
A¹ means NH
R⁴ means OH or NH₂
A² means SH
and q is as defined in claim 5.

15. Compound according to any one of the preceding claims 2 to 14, further characterized that
q means 1 or 2.

16. Compound according to any one of the preceding claims 2 to 15, further characterized that
X means -A¹-CH(NHR³)—(CH₂)_{q}-A²
wherein
A¹ means CO
R³ means H
A² means SH
and q is as defined in claim 5.

17. Compound according to any one of the preceding claims 2 to 16, further characterized that
q means 1.

18. Compound according to any one of the preceding claims 1 to 17, further characterized that
the amino acid residues can be independently chosen from α-amino carbonic acid residues, β-amino carbonic acid residues, aza-amino carbonic acid residues and peptoid-amino carbonic acid residues.

19. Compound according to any one of the preceding claims 1 to 18, further characterized that
the amino acid residues are chosen from α-amino acid residues.

20. Compound according to any one of the preceding claims 1 to 19, further characterized that
the residues Z1 and Z3 or Z1 and Z2 or Z2 and Z3, respectively, are bound to each other through acid-amide bonds —CO—NH—.

21. Compound according to any one of the preceding claims 1 to 20, further characterized that
one or more peptide bonds can be independently modified according to claim 2.

22. Compound according to any one of the preceding claims 1 to 21, further characterized that
the residues Z1 and Z3 or Z1 and Z2 or Z2 and Z3 are bound to each other through —CH₂NH— bonds.

23. Compound according to any one of the preceding claims 1 to 22, further characterized that
direction of the peptide and/or peptido-mimetic sequence is inverted (“retropeptide”).

24. Compound according to any one of the preceding claims 1 to 23, for the treatment of diseases.

25. Support matrix with a surface, to which a compound of any one of the preceding claims 1 to 23 is bound.

26. Support matrix according to claim 25 comprising inorganic or organic, especially polymeric material.

27. Support matrix according to claim 25 or 26 wherein the compound or the compounds of any one of the preceding claims 1 to 23 are chemically bound to the surface of the support matrix, for example to a resin.

28. Support matrix according to claim 25 or 26 wherein the compound or the compounds of any one of the preceding claims 1 to 23 are bound through organic spacers to the surface of the support matrix, for example to a resin.

29. Support matrix according to claim 25 or 26 wherein the compound or the compounds of any one of the preceding claims 1 to 23 are bound through an organic anchoring molecule to the surface of the support matrix, for example to a resin.

30. Support matrix according to claim 25 or 26 wherein the compound or the compounds of any one of the preceding claims 1 to 23 are bound through organic spacers and an organic anchoring molecule to the surface of the support matrix, for example to a resin.

31. Diagnostic device or kit comprising a compound according to any one of the preceding claims 1 to 23, the support matrix according to any one of the claims 25 to 30 and if necessary auxiliary means as well as optionally together with another means a label for a compound.

32. Use of the compound according to any one of the preceding claims 1 to 23 for labeling, detecting, identifying, isolating and purifying FVIII or FVIII-related proteins.

33. A method of detecting, identifying, isolating and purifying FVIII or FVIII-related proteins comprising contacting a sample comprising a FVIII or FVIII-related protein with a matrix comprising an immobilized compound according to any one of the preceding claims 1 to 22, under conditions suitable for binding between FVIII or FVIII-related protein and said compound.

34. The method of claim 33, wherein the compound according to any one of the preceding claims 1 to 23 is immobilized on a support and the support is a polymeric material.

35. The method of claim 33 or 34, wherein the compound according to any one of the preceding claims 1 to 23 is selected from the group consisting of P1, P2, P3, P4, P5, P6, P7, P8, P9, P10, P11, P12, P13, P14, P15, P16, P17, P8, P19, P20, P21, P22, P23, P24 and P25.

36. The method of claim 33 or 34, wherein the polymeric material is a resin.

37. The method according to any one of the preceding claims 33 to 35, wherein the sample is a cell culture supernatant.

38. The method according to any one of the preceding claims 33 to 35, wherein the sample is a body fluid.

39. The method of claim 38, wherein the body fluid comprises blood.

40. A bit or well comprising (1) the compound according to any one of the preceding claims 1 to 23, (2) a matrix comprising a polymeric material, wherein said compound is immobilized on the polymeric material.

41. The bit or well of claim 40, wherein the compound further comprises a label.

42. The method of claim 33 or 34, wherein the compound is immobilized on the polymeric material via a chemically binding to said polymeric material.

43. The method of claim 33 or 34, wherein the peptide or peptido-mimetic is immobilized on the polymeric material via an organic anchoring molecule, which is chemically bound to said polymeric material.

44. The method of claim 33 or 34, wherein the organic anchoring molecule is
-A1-(CH2)p-A2
-A1-CH2-(OCH2CH2)y-O—CH2-A2
-A1-CH2CH2-(OCH2CH2)y-A2
═CR2-(CH2)p-A2
-A1-CH(NHR3)-(CH2)q-A2
═CR2-CH(NHR3)-(CH2)q-A2
-A1-CH(COR4)-(CH2)q-A2
═CR2-CH(COR4)-(CH2)q-A2
wherein A1, A2, R2, R3, R4, p, q and y are as defined in claim 5.

45. The method of claim 33, 34, or 44, wherein the peptide or peptido-mimetic further comprises an organic spacer molecule.

46. The method of claim 45, wherein the organic spacer molecule is selected from one of the following groups
[—NH—(CH2)x-CO]w,
[—NH—(CH2CH2-O-)yCH2-CO]w,
[CO—(CH2)z-CO—],
[NH—(CH2)z-NH—],
[CO—CH2-(OCH2CH2)y-O—CH2-CO—],
[NH—CH2CH2-(OCH2CH2)y-NH—],
or an amino acid, peptide, saccharide or polyether as well as their combinations,
wherein w, x, y and z are as defined in claim 4.
